(19) United States
(12) Patent Application Publication
(10) Pub. No.: US 2023/0255442 A1
(43) Pub. Date: Aug. 17, 2023

(54) CONTINUUM ROBOT APPARATUSES, METHODS, AND STORAGE MEDIUMS

(71) Applicants: Canon U.S.A., Inc., Melville, NY (US); The Brigham and Women's Hospital Inc., Boston, MA (US)

(72) Inventors: Fumitaro Masaki, Brookline, MA (US); Franklin King, Boston, MA (US); Nobuhiko Hata, Newton, MA (US); Brian Ninni, Woburn, MA (US); Takahisa Kato, Brookline, MA (US)

(21) Appl. No.: 18/166,997

(22) Filed: Feb. 9, 2023

Related U.S. Application Data
(60) Provisional application No. 63/309,381, filed on Feb. 11, 2022.

(51) Int. Cl. A61B 1/00; A61B 1/005; A61B 1/01

(52) U.S. Cl. CPC A61B 1/00009; A61B 1/00052; A61B 1/0055; A61B 1/0004; A61B 1/01

(57) ABSTRACT

One or more devices, systems, methods, and storage mediums for performing image correction and/or adjustment are provided herein. Examples of such image correction and/or adjustment include, but are not limited to, correction of a direction to which a tool channel or a camera moves or is bent in a case where a displayed image is rotated. Examples of applications include imaging, evaluating, and diagnosing biological objects, such as, but not limited to, for Gastro-intestinal, cardio, bronchial, and/or ophthalmic applications, and being obtained via one or more optical instruments, such as, but not limited to, optical probes, catheters, endoscopes, and bronchoscopes. Techniques provided herein also improve processing and imaging efficiency while achieving images that are more precise, and also achieve imaging devices, systems, methods, and storage mediums that reduce mental and physical burden and improve ease of use.

### CROSS-REFERENCE TO RELATED APPLICATION(S)

This application relates, and claims priority, to U.S. Prov. Patent Application Ser. No. 63/309,381, filed Feb. 11, 2022, the disclosure of which is incorporated by reference herein in its entirety.

### FIELD OF THE DISCLOSURE

The present disclosure generally relates to imaging and, more particularly, to a continuum robot apparatus, method, and storage medium to implement automatic correction or adjustment of a direction or view to which a tool channel or a camera moves or is bent in a case where a displayed image is rotated. One or more endoscopic, medical, camera, catheter, or imaging devices, systems, and methods and/or storage mediums for use with same, are discussed herein. One or more devices, methods, or storage mediums may be used for medical applications and, more particularly, to steerable, flexible medical devices that may be used for or with guide tools and devices in medical procedures, including, but not limited to, endoscopes, cameras, and catheters.

### BACKGROUND

Endoscopy, bronchoscopy, and other medical procedures facilitate the ability to look inside a body. During such a procedure, a flexible medical tool may be inserted into a patient's body, and an instrument may be passed through the tool to examine or treat an area inside the body. A bronchoscope is an endoscopic instrument to view inside the airways of a patient. Catheters and other medical tools may be inserted through a tool channel in the bronchoscope to provide a pathway to a target area in the patient for diagnosis, planning, medical procedure(s), treatment, etc.

Robotic bronchoscopes may be equipped with a tool channel or a camera and biopsy tools, and may insert/retract the camera and biopsy tools to exchange such components. The robotic bronchoscopes may be used in association with a display system and a control system.

An imaging device, such as a camera, may be placed in the bronchoscope to capture images inside the patient, and a display or monitor may be used to view the captured images. The display system may display, on the monitor, an image or images captured by the camera, and the display system may have a display coordinate used for displaying the captured image or images. In addition, the control system may control a moving direction of the tool channel or the camera. For example, the tool channel or the camera may be bent according to a control by the control system. The control system may have an operational controller (such as, but not limited to, a joystick, a gamepad, a controller, an input device, etc.).

Calibration may take place between movement of the camera and movement of a captured image on the display. If the captured image is rotated on a display coordinate after the calibration is performed, a relationship between positions of a displayed image and positions of the monitor is changed. On the other hand, the tool channel or the camera may move or may be bent in the same way regardless of the rotation of the displayed image when, or in a case where, a particular command is received to move or change position, for example, a command to let the tool channel, the camera, or a capturing direction of the camera move or change position. Such a move or a change in position may cause a change of a relationship between the positions of the monitor and a direction to which the tool channel or the camera moves on the monitor according to a particular command, for example, tilting a joystick (or other operational controller) up, down, right, or left. For example, when the calibration is performed, by tilting the joystick upward, the tool channel or the camera may bend to a direction corresponding to a top of the monitor. However, after the captured image on the display is rotated, by tilting the joystick upward, the tool channel or the camera may not be bent to the direction corresponding to the top of the monitor but may be bent to a direction diagonally upward of the monitor. Such a situation may complicate user interaction between the camera and the monitor.

Physicians may rotate the camera or captured image(s) so that a display layout of airways in the captured image(s) matches to a preset or predetermined layout (e.g., a typical layout, a preferred or desired layout, etc.). For example, if right and left main bronchus in a captured image or images are not displayed horizontally on the display, a physician may rotate the camera so that the right and left main bronchus are displayed horizontally on the monitor. However, depending on the structure or control of a particular device being used (e.g., a robotic bronchoscope, a catheter, an endoscope, an imaging device, a medical device, etc.), rotational orientations or behaviors of that device may be different than other devices that physicians are used to using, such as, but not limited to, conventional endoscopes, conventional bronchoscopes, endoscopes or bronchoscopes that the physician or user of the endoscopes or bronchoscopes is familiar or has experience with, etc. In a case where a first device has different controls, structure(s), and/or behaviors (e.g., rotational orientation behaves differently) than a second device having preset or predetermined control(s), structure(s) and/or behavior(s), use of the first device may potentially confuse physicians familiar with, or having experience using, the second device. For example, in a case where the first device and second device have different navigation controls and/or rotational behaviors or orientations, a physician may be confused when navigating or controlling the first device in comparison to the second device.

As such, there is a need for devices, systems, methods, and/or storage mediums that avoid any confusion that may be caused by differences in navigation and/or orientation (e.g., rotational) control between a first device or system and a second device or system and that reduce or avoid mental burden and/or physical labor of a user (e.g., a physician) of the first device or second device to compensate for the navigation and/or orientation (e.g., rotational orientation) differences.

Accordingly, it would be desirable to provide at least one imaging, optical, or control device, system, method, and storage medium for controlling one or more endoscopic or imaging devices or systems, for example, by implementing automatic correction or adjustment of a direction or view to which a tool channel or a camera moves or is bent in a case where a displayed image is rotated.

### SUMMARY

Accordingly, it is a broad object of the present disclosure to provide imaging (e.g., computed tomography (CT), Magnetic Resonance Imaging (MRI), etc.) apparatuses, systems, methods, and storage mediums for using and/or controlling a correction or adjustment method in one or more apparatuses or systems (e.g., an imaging apparatus or system, an endoscopic imaging device or system, etc.). The correction or adjustment may be made to a direction or view of the one or more apparatuses or systems.

One or more embodiments of the present disclosure avoid the aforementioned issues by providing a simple and fast method or methods that normalizes displaying or viewing images, and/or controlling navigation and/or orientation (e.g., rotational orientation), of a first apparatus or system (e.g., a catheter, a camera, etc.) as compared with displaying or viewing images, and/or controlling navigation and/or orientation, of a second apparatus or system having different navigation and/or rotational orientations or behaviors than that of the first apparatus or system. As such, physicians or other users of the first apparatus or system may have reduced or saved labor and/or mental burden using the first apparatus or system, regardless of any difference between the first apparatus or system or any other apparatus or system (e.g., the second apparatus or system). In one or more embodiments of the present disclosure, a labor of a user to rotate the camera to acquire a typical camera view is saved or reduced. In one or more embodiments of the present disclosure, a discomfort or any confusion because of a difference of a type of a catheter or other imaging device from another catheter or other imaging device that a user has experience with is reduced or avoided. In one or more embodiments, a user may check a captured image or images of a target or object (e.g., inside of a lung) as if the user is using any type of apparatus or system (e.g., regardless of whether the user is using a first apparatus or system (e.g., a first endoscope) or a second apparatus or system (e.g., a second endoscope) having different viewing and/or navigation controls or orientations (and/or other structural or control differences), the user may successfully and easily view the captured image or images).

In one or more embodiments of the present disclosure, an apparatus or system may include one or more processors that operate to: receive a captured image or images captured by a first imaging device at a first position, obtain or determine an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device, and adjust or correct the captured image or images based on the estimated image or images. In one or more embodiments, the apparatus or system may include a display to display the adjusted or corrected image or images.

In one or more embodiments, an apparatus or system may include a display to display the adjusted or corrected image or images. In one or more embodiments, an apparatus or system may further include: a receiver that operates to receive the captured image or images captured by the first imaging device at the first position, the first imaging device being a first endoscope, and transmit the capture image or images to the one or more processors such that the one or more processors receive the captured image or images; a controller as being part of the one or more processors, the controller operating to obtain or determine the estimated image or images that would have been captured by the second imaging device at the first position, the second imaging device being a second endoscope; and a display controller as being part of the one or more processors, the display controller or the one or more processors operating to provide an image or images for display, the image or images for display being based on the captured image or images and information for the estimated image or images. In one or more embodiments, the second endoscope may have a lower degree of freedom from a bendable degree of freedom of the first endoscope. In one or more embodiments, the first endoscope may be a camera deployed at a tip of a steerable catheter and may be bent with the steerable catheter, and/or the camera may be detachably attached to, or removably inserted into, the steerable catheter. In one or more embodiments, the second endoscope may be a virtual endoscope or is represented by a preset or predetermined data profile. In one or more embodiments, the first endoscope may have a bending section that operates to bend three-dimensionally and/or to bend on two or more planes. In one or more embodiments, the second endoscope may have a bending section that can bend only on one plane. In one or more embodiments, the image or images for display may be the captured image or images that are rotated so that an orientation of the image or images for display corresponds to an orientation of the estimated image or images, and the display controller or the one or more processors may display the image or images for display on a display.

In one or more embodiments, the image or images for display may include the captured image or images and an additional image or images, the additional image or images being the captured image or images that are rotated based on the information for the estimated image or images. The captured image or images and the additional image or images may be displayed at the same time. The display controller or the one or more processors may further operate to display the additional image or images in accordance with a user instruction to display the additional image or images on the display. The display controller or the one or more processors may further operate to switch the image or images for display from the captured image or images to the additional image or images. In one or more embodiments, an apparatus or system may further include: an interface configured to receive a command to bend the first endoscope, wherein the display controller or the one or more processors may further operate to temporarily suspend the rotation of the captured image or images while the interface is receiving the command. In one or more embodiments, an apparatus or system may further include: an interface configured to receive a command to bend the first endoscope, wherein the display controller or the one or more processors further operate to restrict an amount of rotation of the captured image or images captured by the first endoscope in a case where the rotation angle of the captured image or images in accordance with the received command is larger than a predetermined rotation angle. In one or more embodiments, an apparatus or system may further include: an interface configured to receive a command to bend the first endoscope, wherein the interface receives the command corresponding to a bending direction or a twisting amount of the second endoscope.

In one or more embodiments, an apparatus or system may further include: a steerable catheter with at least one bending section and an endoscope camera; and an actuation unit or a driver that operates to bend the bending section, wherein the controller or the one or more processors may further operate to: receive one or more control commands or instructions for a bending amount and a bending plane orientation; send the one or more commands or instructions to the actuation unit or the driver to bend the bending section; and receive one or more endoscopic images and display the one or more endoscopic images on a display. In one or more embodiments, one or more of the following may occur or exist: (i) the imaging device may further include an operational controller or joystick that operates to issue or input the one or more commands or instructions to the controller or the one or more processors; (ii) the imaging device may further include a display to display the one or more endoscopic images, or the imaging device may further include a display to display the one or more endoscopic images where the display has a reference direction; (iii) the controller or the one or more processors may further operate to store the bending plane orientation in the one or more control commands or instructions in relation to the one or more endoscopic images during navigation; (iv) the controller or the one or more processors may further operate to rotate a current endoscopic image to an orientation where a bending plane orientation stored at the last moment or stored last is aligned to a reference direction of a display of the imaging device; and/or (v) the imaging device may further include an operational controller or joystick that operates to issue or input the one or more commands or instructions to the controller or the one or more processors, and the operational controller or joystick operates to be controlled by a user of the imaging device. One or more embodiments may further include an operational controller or joystick, the operational controller or joystick having a rotation controller and a bending controller, wherein one or more of the following may occur: the rotation controller operates to issue a control command or instruction of or for the bending plane orientation; the bending controller operates to issue a control command or instruction of or for the bending amount; and/or in a case where the controller or the one or more processors further operate to rotate a current endoscopic image to an orientation where a bending plane orientation stored at the last moment or stored last is aligned to a reference direction of a display of the imaging device, the controller or the one or more processors further operate to rotate the current endoscopic image as the rotation controller issues the control command or instruction of or for the bending plane orientation. In one or more embodiments, an apparatus or system may further include: a steerable catheter with at least one bending section and an endoscope camera; an operational controller or joystick that operates to issue or input one or more commands or instructions of a bending amount and a bending plane orientation into the imaging device; and a tracking device that operates to track a real-time bending plane orientation, wherein the controller or the one or more processors may operate to receive one or more endoscopic images and the one or more commands or instructions. In one or more embodiments, one or more of the following may occur or exist: (i) the imaging device further comprises a display that operates to display the one or more endoscopic images; (ii) the controller or the one or more processors further operate to recode the bending plane orientation in relation to the one or more endoscopic images during navigation; and/or (iii) the imaging device further comprises a display that operates to display the one or more endoscopic images, the display having a reference direction, where the controller or the one or more processors further operate to rotate the current endoscopic image to an orientation where the real-time bending plane orientation from the tracking device is aligned to the reference direction of the display.

In one or more embodiments, a control apparatus may include: a receiver that operates to receive a captured image or images captured by a first endoscope at a first position; a controller that operates to obtain or determine an estimated image or images that would have been captured by a second endoscope at the first position, the second endoscope having preset or predetermined navigation and/or viewing controls or orientations, and/or preset or predetermined structural features, that are different than navigation and/or viewing controls or orientations, and/or structural features, of the first endoscope; and a display controller that operates to provide an image or images for display, the image or images for display being based on the captured image or images and information for the estimated image or images. In one or more embodiments, the second endoscope may have a lower degree of freedom from a bendable degree of freedom of the first endoscope. In one or more embodiments, the first endoscope may be a camera deployed at a tip of a steerable catheter and may be bent with the steerable catheter, and/or the camera may be detachably attached to, or removably inserted into, the steerable catheter. In one or more embodiments, the second endoscope may be a virtual endoscope or may be represented by a preset or predetermined data profile. In one or more embodiments, the first endoscope may have a bending section that operates to bend three-dimensionally and/or to bend on two or more planes. In one or more embodiments, the second endoscope may have a bending section that can bend only on one plane. In one or more embodiments, the image or images for display may be the captured image or images that are rotated so that an orientation of the image or images for display corresponds to an orientation of the estimated image or images, and the display controller may display the image or images for display on a display. In one or more embodiments, the image or images for display may include the captured image or images and an additional image or images, the additional image or images being the captured image or images that are rotated based on the information for the estimated image or images. In one or more embodiments, the captured image or images and the additional image or images may be displayed at the same time. In one or more embodiments, the display controller may further operate to display the additional image or images in accordance with a user instruction to display the additional image or images on the display. In one or more embodiments, the display controller may further operate to switch the image or images for display from the captured image or images to the additional image or images. One or more embodiments of a control apparatus may further include: an interface configured to receive a command to bend the first endoscope, wherein the display controller further operates to temporarily suspend the rotation of the captured image or images while the interface is receiving the command. One or more embodiments of a control apparatus may further include: an interface configured to receive a command to bend the first endoscope, wherein the display controller further operates to restrict an amount of rotation of the captured image or images captured by the first endoscope in a case where the rotation angle of the captured image or images in accordance with the received command is larger than a predetermined rotation angle. One or more embodiments of a control apparatus may further include: an interface configured to receive a command to bend the first endoscope, wherein the interface receives the command corresponding to a bending direction or a twisting amount of the second endoscope.

In one or more embodiments, an endoscope system may include: a steerable catheter with at least one bending section and an endoscope camera; an actuation unit or a driver that operates to bend the bending section; and a controller or one or more processors that operate to: receive one or more control commands or instructions for a bending amount and a bending plane orientation; send the one or more commands or instructions to the actuation unit or driver to bend the bending section; and receive one or more endoscopic images and display the one or more endoscopic images on a display. In one or more embodiments, one or more of the following may exist or may occur: (i) the endoscope system further comprises an operational controller or joystick that operates to issue or input the one or more commands or instructions to the controller or the one or more processors; (ii) the endoscope system further includes a display to display the one or more endoscopic images, or the endoscope system further includes a display to display the one or more endoscopic images where the display has a reference direction; (iii) the controller or the one or more processors further operate to store the bending plane orientation in the one or more control commands or instructions in relation to the one or more endoscopic images during navigation; (iv) the controller or the one or more processors further operate to rotate a current endoscopic image to an orientation where a bending plane orientation stored at the last moment or stored last is aligned to a reference direction of a display of the endoscope system; and/or (v) the endoscope system further comprises an operational controller or joystick that operates to issue or input the one or more commands or instructions to the controller or the one or more processors, and the operational controller or joystick operates to be controlled by a user of the endoscope system. In one or more embodiments, the endoscope system may further include an operational controller or joystick, the operational controller or joystick having a rotation controller and a bending controller, wherein one or more of the following: the rotation controller operating to issue a control command or instruction of or for the bending plane orientation; the bending controller operating to issue a control command or instruction of or for the bending amount; and/or in a case where the controller or the one or more processors further operate to rotate a current endoscopic image to an orientation where a bending plane orientation stored at the last moment or stored last is aligned to a reference direction of a display of the endoscope system, the controller or the one or more processors further operate to rotate the current endoscopic image as the rotation controller issues the control command or instruction of or for the bending plane orientation.

In one or more embodiments, an endoscope system may include: a steerable catheter with at least one bending section and an endoscope camera; an operational controller or joystick that operates to issue or input one or more commands or instructions of a bending amount and a bending plane orientation into the endoscope system; a tracking device that operates to track a real-time bending plane orientation; and a controller or one or more processors that operate to receive one or more endoscopic images and the one or more commands or instructions. In one or more embodiments, one or more of the following may exist or may occur: (i) the endoscope system further comprises a display that operates to display the one or more endoscopic images; (ii) the controller or the one or more processors further operate to recode the bending plane orientation in relation to the one or more endoscopic images during navigation; and/or (iii) the endoscope system further comprises a display that operates to display the one or more endoscopic images, the display having a reference direction, where the controller or the one or more processors further operate to rotate the current endoscopic image to an orientation where the real-time bending plane orientation from the tracking device is aligned to the reference direction of the display.

In one or more embodiments, a method for performing image correction and/or adjustment may include: receiving a captured image or images captured by a first imaging device at a first position; obtaining or determining an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device; adjusting or correcting the captured image or images based on the estimated image or images; and displaying the adjusted or corrected image or images on a display. In one or more embodiments, the first imaging device may be a first endoscopic device and the second image device may be a second endoscopic device.

In one or more embodiments, a non-transitory computer-readable storage medium may store at least one program for causing a computer to execute a method for performing image correction and/or adjustment, the method comprising: receiving a captured image or images captured by a first imaging device at a first position; obtaining or determining an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device; adjusting or correcting the captured image or images based on the estimated image or images; and displaying the adjusted or corrected image or images on a display.

In accordance with one or more embodiments of the present disclosure, apparatuses and systems, and methods and storage mediums for performing correction(s) and/or adjustment(s) to a direction or view may operate to characterize biological objects, such as, but not limited to, blood, mucus, tissue, etc.

One or more embodiments of the present disclosure may be used in clinical application(s), such as, but not limited to, intervascular imaging, intravascular imaging, bronchoscopy, atherosclerotic plaque assessment, cardiac stent evaluation, intracoronary imaging using blood clearing, balloon sinuplasty, sinus stenting, arthroscopy, ophthalmology, ear research, veterinary use and research, etc.

In accordance with at least another aspect of the present disclosure, one or more technique(s) discussed herein may be employed as or along with features to reduce the cost of at least one of manufacture and maintenance of the one or more apparatuses, devices, systems, and storage mediums by reducing or minimizing a number of optical and/or processing components and by virtue of the efficient techniques to cut down cost (e.g., physical labor, mental burden, fiscal cost, time and complexity, etc.) of use/manufacture of such apparatuses, devices, systems, and storage mediums.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods, and one or more storage mediums using imaging adjustment or correction and/or other technique(s) are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 illustrates at least one embodiment of an imaging or endoscopic apparatus or system in accordance with one or more aspects of the present disclosure;

FIG. 2 is a schematic diagram showing at least one embodiment an imaging or endoscopic apparatus or system in accordance with one or more aspects of the present disclosure;

FIG. 3 is a schematic diagram showing at least one embodiment console or computer that may be used with one or more correction or adjustment imaging technique(s) in accordance with one or more aspects of the present disclosure;

FIGS. 4A-4B illustrate at least one embodiment example of a continuum robot and/or medical device that may be used with one or more correction or adjustment imaging technique(s) in accordance with one or more aspects of the present disclosure;

FIG. 5 is a schematic diagram showing at least one embodiment an imaging or continuum robot apparatus or system in accordance with one or more aspects of the present disclosure;

FIG. 6 is a flowchart of at least one embodiment of a method for planning an operation of at least one embodiment of a continuum robot apparatus or system in accordance with one or more aspects of the present disclosure;

FIG. 7 is a flowchart of at least one embodiment of a method for planning an operation of at least one embodiment of a continuum robot apparatus or system in accordance with one or more aspects of the present disclosure;

FIG. 8A shows insertion and removal details for an endoscope that is bendable in a single plane in accordance with one or more aspects of the present disclosure;

FIG. 8B shows insertion and removal details for an endoscope that is bendable in multiple planes in accordance with one or more aspects of the present disclosure;

FIG. 9 illustrates at least one embodiment of an endoscope using adjustment or correction technique(s) in accordance with one or more aspects of the present disclosure;

FIG. 10 is a flowchart of at least one embodiment of a method for performing adjustment or correction of an image, view, or display in accordance with one or more aspects of the present disclosure;

FIG. 11 illustrates at least one embodiment of a planning of a route for an endoscope that is bendable on only one plane in accordance with one or more aspects of the present disclosure;

FIG. 12 is a flowchart of at least one embodiment of a method for performing adjustment or correction of an image, view, or display using pre-planned or predetermined data in accordance with one or more aspects of the present disclosure;

FIG. 13 is a diagrammatic illustration of at least one embodiment of a display of an image captured by a first endoscope that can bend in multiple planes and of an estimated or theoretical image that would have been captured by a second endoscope that can bend in only one plane in accordance with one or more aspects of the present disclosure;

FIGS. 14A-14B illustrate at least one embodiment of a display of an estimated or theoretical image that would have been captured by a second endoscope that can bend in only one plane and a display that operates to control a first endoscope that can bend in more than one plane while viewing the display of the estimated or theoretical image, respectively, in accordance with one or more aspects of the present disclosure;

FIG. 15 illustrates a diagram of a continuum robot that may be used with one or more adjustment or correction technique(s) or method(s) in accordance with one or more aspects of the present disclosure;

FIG. 16 illustrates a block diagram of at least one embodiment of a continuum robot in accordance with one or more aspects of the present disclosure;

FIG. 17 illustrates a block diagram of at least one embodiment of a controller in accordance with one or more aspects of the present disclosure;

FIG. 18 is a flowchart of at least one embodiment of a method for performing an automatic correction of a bending direction of a bronchoscope that may be used in accordance with one or more aspects of the present disclosure;

FIG. 19 is a flowchart of at least a further embodiment of a method for performing an automatic correction of a bending direction of a bronchoscope that may be used in accordance with one or more aspects of the present disclosure;

FIG. 20 shows a schematic diagram of an embodiment of a computer that may be used with one or more embodiments of an apparatus or system or one or more methods discussed herein in accordance with one or more aspects of the present disclosure; and

FIG. 21 shows a schematic diagram of another embodiment of a computer that may be used with one or more embodiments of an imaging apparatus or system or methods discussed herein in accordance with one or more aspects of the present disclosure.

### DETAILED DESCRIPTION OF THE PRESENT DISCLOSURE

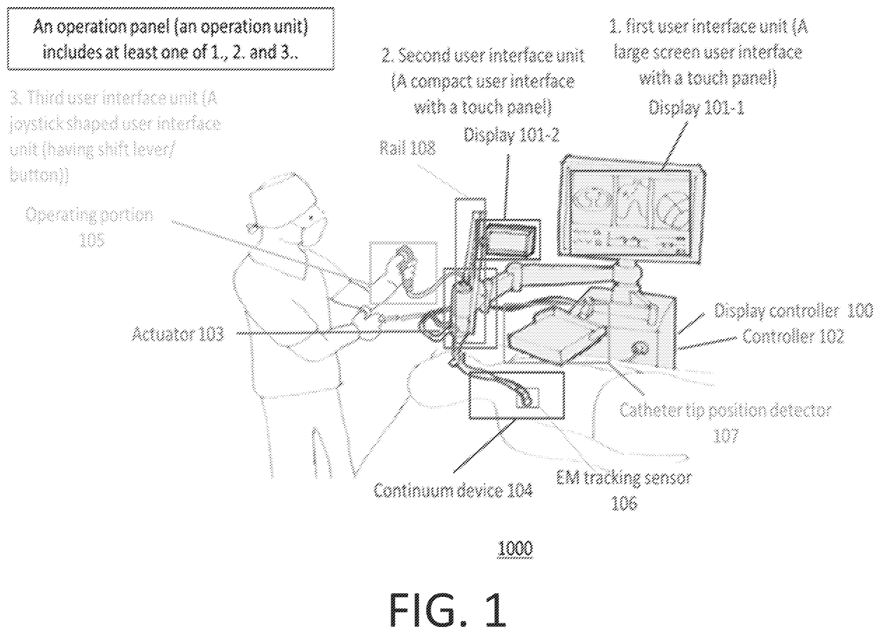

One or more devices, systems, methods and storage mediums for viewing, imaging, and/or characterizing tissue, or an object or sample, using one or more imaging techniques or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure are described diagrammatically and visually in FIGS. 1 through 21.

One or more embodiments of the present disclosure avoid the aforementioned issues by providing a simple and fast method or methods that normalizes displaying or viewing images, and/or controlling navigation and/or orientation (e.g., rotational orientation), of a first apparatus or system (e.g., a catheter, a camera, etc.) as compared with displaying or viewing images, and/or controlling navigation and/or orientation, of a second apparatus or system having different navigation and/or rotational orientations or behaviors than that of the first apparatus or system. As such, physicians or other users of the first apparatus or system may have reduced or saved labor and/or mental burden using the first apparatus or system, regardless of any difference between the first apparatus or system or any other apparatus or system (e.g., the second apparatus or system). In one or more embodiments of the present disclosure, a labor of a user to rotate the camera to acquire a typical camera view is saved or reduced. In one or more embodiments of the present disclosure, a discomfort or any confusion because of a difference of a type of a catheter or other imaging device from another catheter or other imaging device that a user has experience with is reduced or avoided. In one or more embodiments, a user may check a captured image or images of a target or object (e.g., inside of a lung) as if the user is using any type of apparatus or system (e.g., regardless of whether the user is using a first apparatus or system (e.g., a first endoscope) or a second apparatus or system (e.g., a second endoscope) having different viewing and/or navigation controls or orientations (and/or other structural or control differences), the user may successfully and easily view the captured image or images).

In one or more embodiments of the present disclosure, an apparatus or system may include one or more processors that operate to: receive a captured image or images captured by a first imaging device at a first position, obtain or determine an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device, and adjust or correct the captured image or images based on the estimated image or images. In one or more embodiments, the apparatus or system may include a display to display the adjusted or corrected image or images.

In one or more embodiments, a control apparatus may include: a receiver that operates to receive a captured image or images captured by a first endoscope at a first position; a controller that operates to obtain or determine an estimated image or images that would have been captured by a second endoscope at the first position, the second endoscope having preset or predetermined navigation and/or viewing controls or orientations, and/or preset or predetermined structural features, that are different than navigation and/or viewing controls or orientations, and/or structural features, of the first endoscope; and a display controller that operates to provide an image or images for display, the image or images for display being based on the captured image or images and information for the estimated image or images. In one or more embodiments, the second endoscope may have a lower degree of freedom from a bendable degree of freedom of the first endoscope. In one or more embodiments, the first endoscope may be a camera deployed at a tip of a steerable catheter and may be bent with the steerable catheter, and/or the camera may be detachably attached to, or removably inserted into, the steerable catheter. In one or more embodiments, the second endoscope may be a virtual endoscope or may be represented by a preset or predetermined data profile. In one or more embodiments, the first endoscope may have a bending section that operates to bend three-dimensionally and/or to bend on two or more planes. In one or more embodiments, the second endoscope may have a bending section that can bend only on one plane. In one or more embodiments, the image or images for display may be the captured image or images that are rotated so that an orientation of the image or images for display corresponds to an orientation of the estimated image or images, and the display controller may display the image or images for display on a display. In one or more embodiments, the image or images for display may include the captured image or images and an additional image or images, the additional image or images being the captured image or images that are rotated based on the information for the estimated image or images. In one or more embodiments, the captured image or images and the additional image or images may be displayed at the same time. In one or more embodiments, the display controller may further operate to display the additional image or images in accordance with a user instruction to display the additional image or images on the display. In one or more embodiments, the display controller may further operate to switch the image or images for display from the captured image or images to the additional image or images. One or more embodiments of a control apparatus may further include: an interface configured to receive a command to bend the first endoscope, wherein the display controller further operates to temporarily suspend the rotation of the captured image or images while the interface is receiving the command. One or more embodiments of a control apparatus may further include: an interface configured to receive a command to bend the first endoscope, wherein the display controller further operates to restrict an amount of rotation of the captured image or images captured by the first endoscope in a case where the rotation angle of the captured image or images in accordance with the received command is larger than a predetermined rotation angle. One or more embodiments of a control apparatus may further include: an interface configured to receive a command to bend the first endoscope, wherein the interface receives the command corresponding to a bending direction or a twisting amount of the second endoscope.

In one or more embodiments, an endoscope system may include: a steerable catheter with at least one bending section and an endoscope camera; an actuation unit or a driver that operates to bend the bending section; and a controller or one or more processors that operate to: receive one or more control commands or instructions for a bending amount and a bending plane orientation; send the one or more commands or instructions to the actuation unit or driver to bend the bending section; and receive one or more endoscopic images and display the one or more endoscopic images on a display. In one or more embodiments, one or more of the following may exist or may occur: (i) the endoscope system further comprises an operational controller or joystick that operates to issue or input the one or more commands or instructions to the controller or the one or more processors; (ii) the endoscope system further includes a display to display the one or more endoscopic images, or the endoscope system further includes a display to display the one or more endoscopic images where the display has a reference direction; (iii) the controller or the one or more processors further operate to store the bending plane orientation in the one or more control commands or instructions in relation to the one or more endoscopic images during navigation; (iv) the controller or the one or more processors further operate to rotate a current endoscopic image to an orientation where a bending plane orientation stored at the last moment or stored last is aligned to a reference direction of a display of the endoscope system; and/or (v) the endoscope system further comprises an operational controller or joystick that operates to issue or input the one or more commands or instructions to the controller or the one or more processors, and the operational controller or joystick operates to be controlled by a user of the endoscope system. In one or more embodiments, the endoscope system may further include an operational controller or joystick, the operational controller or joystick having a rotation controller and a bending controller, wherein one or more of the following: the rotation controller operating to issue a control command or instruction of or for the bending plane orientation; the bending controller operating to issue a control command or instruction of or for the bending amount; and/or in a case where the controller or the one or more processors further operate to rotate a current endoscopic image to an orientation where a bending plane orientation stored at the last moment or stored last is aligned to a reference direction of a display of the endoscope system, the controller or the one or more processors further operate to rotate the current endoscopic image as the rotation controller issues the control command or instruction of or for the bending plane orientation.

In one or more embodiments, an endoscope system may include: a steerable catheter with at least one bending section and an endoscope camera; an operational controller or joystick that operates to issue or input one or more commands or instructions of a bending amount and a bending plane orientation into the endoscope system; a tracking device that operates to track a real-time bending plane orientation; and a controller or one or more processors that operate to receive one or more endoscopic images and the one or more commands or instructions. In one or more embodiments, one or more of the following may exist or may occur: (i) the endoscope system further comprises a display that operates to display the one or more endoscopic images; (ii) the controller or the one or more processors further operate to recode the bending plane orientation in relation to the one or more endoscopic images during navigation; and/or (iii) the endoscope system further comprises a display that operates to display the one or more endoscopic images, the display having a reference direction, where the controller or the one or more processors further operate to rotate the current endoscopic image to an orientation where the real-time bending plane orientation from the tracking device is aligned to the reference direction of the display.

In one or more embodiments, a method for performing image correction and/or adjustment may include: receiving a captured image or images captured by a first imaging device at a first position; obtaining or determining an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device; adjusting or correcting the captured image or images based on the estimated image or images; and displaying the adjusted or corrected image or images on a display. In one or more embodiments, the first imaging device may be a first endoscopic device and the second image device may be a second endoscopic device.

In one or more embodiments, a non-transitory computer-readable storage medium may store at least one program for causing a computer to execute a method for performing image correction and/or adjustment, the method comprising: receiving a captured image or images captured by a first imaging device at a first position; obtaining or determining an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device; adjusting or correcting the captured image or images based on the estimated image or images; and displaying the adjusted or corrected image or images on a display.

In one or more embodiments the first imaging device or the first endoscope may include bending on two or more planes (e.g., three-dimensional bending) and torqueing such that the first imaging device or the first endoscope achieves improved steering, consistent or reliable orientation (e.g., rotational orientation, orientation inside a catheter, etc.), and imaging while reducing or avoiding any confusion to a user (e.g., a physician) of the first imaging device or the first endoscope (as compared to a second imaging device or endoscope that does not include three-dimensional bending, as compared to a second imaging device or endoscope that does not include bending on two or more planes, as compared to a second imaging device or endoscope that does not include torqueing, etc.).

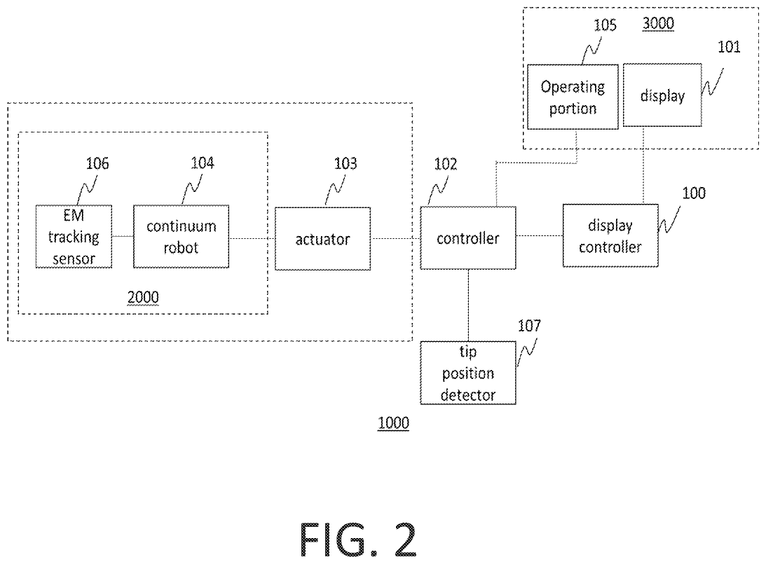

As shown in FIGS. 1-4 of the present disclosure, one or more embodiments of a system 1000 for performing image adjustment and/or correction (e.g., for a continuum robot) may include one or more of the following: a display controller 100, a display 101-1, a display 101-2, a controller 102, an actuator 103, a continuum device 104, an operating portion 105, an EM tracking sensor 106, a catheter tip position detector 107, and a rail 108 (for example, as shown in at least FIGS. 1-2). The system 1000 may include one or more processors, such as, but not limited to, a display controller 100, a controller 102, a CPU 120, a controller 50, a CPU 51, a console or computer 1200 or 1200′, a CPU 1201, any other processor or processors discussed herein, etc., that operate to execute a software program and to control display of a navigation screen on one or more displays 101. The one or more processors (e.g., the display controller 100, the controller 102, the CPU 120, the controller 50, the CPU 51, the console or computer 1200 or 1200′, the CPU 1201, any other processor or processors discussed herein, etc.) may generate a three dimensional (3D) model of a structure (for example, a branching structure like airway of lungs of a patient, an object to be imaged, tissue to be imaged, etc.) based on images, such as, but not limited to, CT images, MRI images, etc. Alternatively, the 3D model may be received by the one or more processors (e.g., the display controller 100, the controller 102, the CPU 120, the controller 50, the CPU 51, the console or computer 1200 or 1200′, the CPU 1201, any other processor or processors discussed herein, etc.) from another device. A two dimensional (2D) model may be used instead of 3D model in one or more embodiments. The 2D or 3D model may be generated before a navigation starts. Alternatively, the 2D or 3D model may be generated in real-time (in parallel with the navigation). In the one or more embodiments discussed herein, examples of generating a model of branching structure are explained. However, the models may not be limited to a model of branching structure. For example, a model of a route direct to a target may be used instead of the branching structure. Alternatively, a model of a broad space may be used, and the model may be a model of a place or a space where an observation or a work is performed by using a continuum robot 104 explained below.

While not limited to such a configuration, the display controller 100 may acquire position information of the continuum robot 104 from a controller 102. Alternatively, the display controller 100 may acquire the position information directly from a tip position detector 107. The continuum robot 104 may be a catheter device. The continuum robot 104 may be attachable/detachable to the actuator 103, and the continuum robot 104 may be disposable.

In one or more embodiments, the one or more processors, such as the display controller 100, may generate and output a navigation screen to the one or more displays 101-1, 101-2 based on the 3D model and the position information by executing the software. The navigation screen indicates a current position of the continuum robot 104 on the 3D model. By the navigation screen, a user can recognize the current position of the continuum robot 104 in the branching structure.

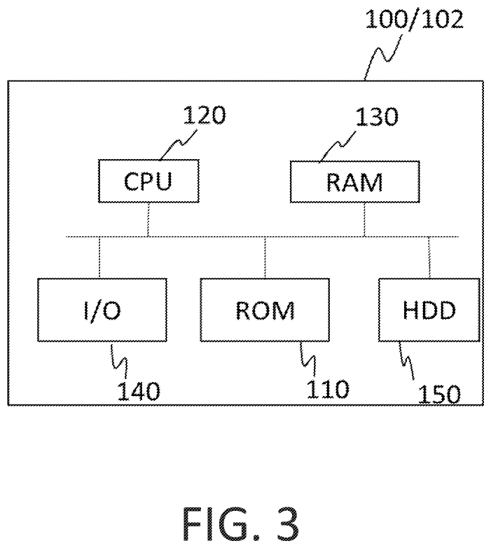

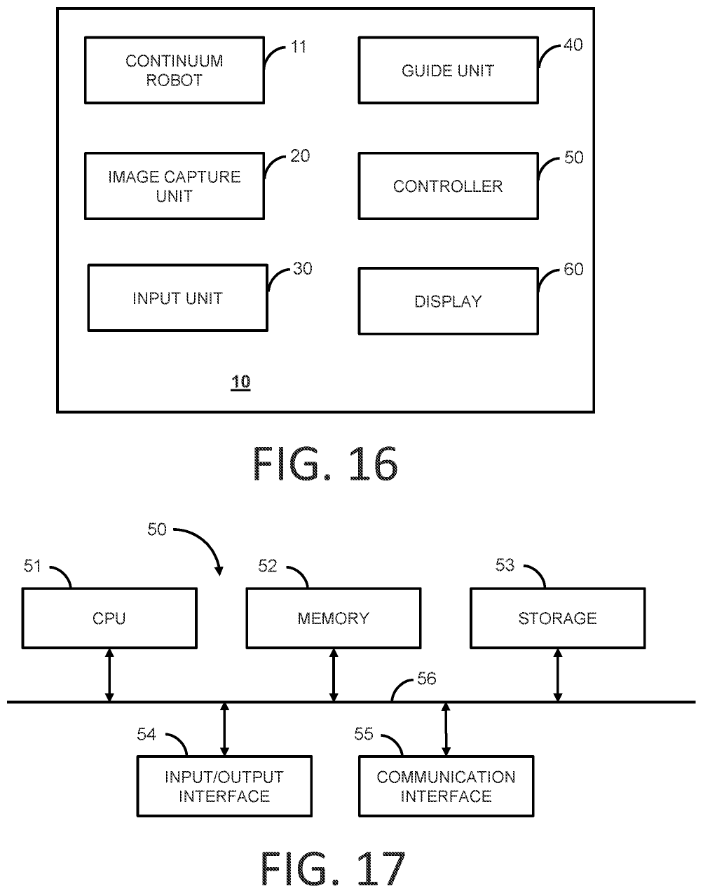

In one or more embodiments, the one or more processors, such as, but not limited to, the display controller 100 and/or the controller 102, may include, as shown in FIG. 3, at least one storage Read Only Memory (ROM) 110, at least one central processing unit (CPU) 120, at least one Random Access Memory (RAM) 130, at least one input and output (I/O) interface 140 and at least one Hard Disc Drive (HDD) 150. A Solid State Drive (SSD) may be used instead of HDD 150. In one or more additional embodiments, the one or more processors, and/or the display controller 100 and/or the controller 102, may include structure as shown in FIGS. 16-17 and 20-21 as further discussed below.

The ROM 110 and/or HDD 150 operate to store the software in one or more embodiments. The RAM 130 may be used as a work memory. The CPU 120 may execute the software program developed in the RAM 130. The I/O 140 operates to input the positional information to the display controller 100 and to output information for displaying the navigation screen to the one or more displays 101-1, 101-2. In the embodiments below, the navigation screen may be generated by the software program. In one or more other embodiments, the navigation screen may be generated by a firmware.

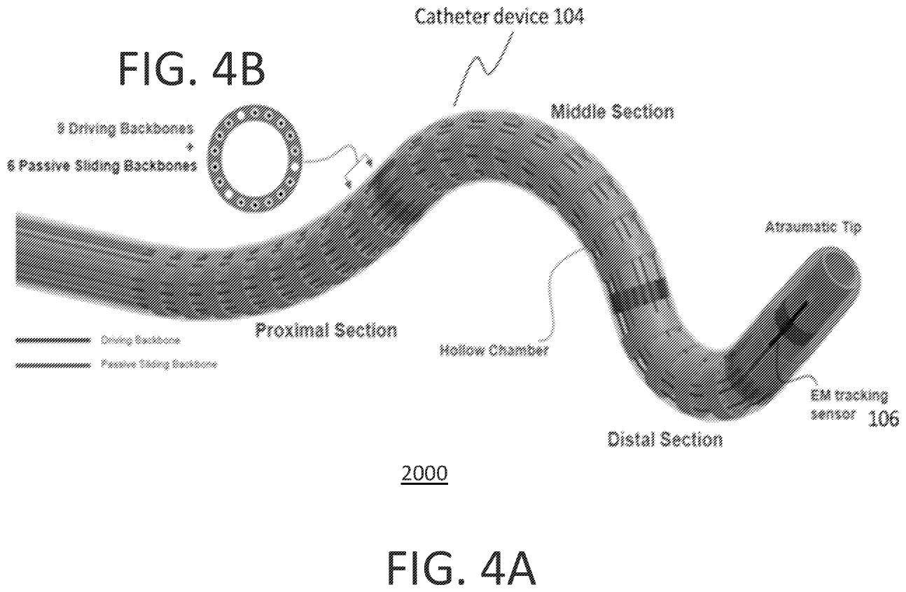

FIGS. 4A-4B show at least one embodiment of a continuum robot 104 that may be used in the system 1000 or any other system discussed herein. The continuum robot 104 may include a proximal section, a middle section, and a distal section, and each of the sections may be bent by a plurality of driving wires (driving liner members, such as a driving backbone or backbones). In one or more embodiments, the continuum robot may be a catheter device 104. The posture of the catheter device 104 may be supported by supporting wires (supporting liner members, for example, passive sliding backbones). The driving wires may be connected to the actuator 103. The actuator 103 may include one or more motors and drives for each of the sections of the catheter 104 by pushing and/or pulling the driving wires (driving backbones). The actuator 103 may proceed or retreat along a rail 108 (e.g., to translate the actuator 103, the continuum robot/catheter 104, etc.), and the actuator 103 and continuum robot 104 may proceed or retreat in and out of the patient's body or other target, object, or specimen (e.g., tissue). As shown in FIG. 4B, the catheter device 104 may include a plurality of driving backbones and may include a plurality of passive sliding backbones. In one or more embodiments, the catheter device 104 may include at least nine (9) driving backbones and at least six (6) passive sliding backbones. The catheter device 104 may include an atraumatic tip at the end of the distal section of the catheter device 104.

One or more embodiments of the catheter/continuum robot 104 may include an electro-magnetic (EM) tracking sensor 106. One or more other embodiments of the catheter/continuum robot 104 may not include or use the EM tracking sensor 106. The electro-magnetic tracking sensor (EM tracking sensor) 106 may be attached to the tip of the continuum robot 104. In this embodiment, a robot 2000 may include the continuum robot 104 and the EM tracking sensor 106 (as seen diagrammatically in FIG. 2), and the robot 2000 may be connected to the actuator 103.

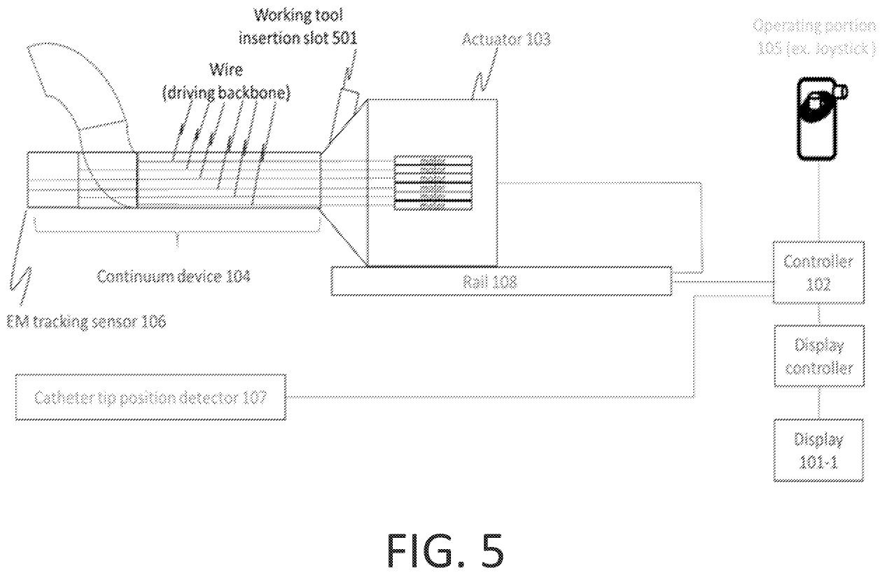

One or more devices or systems, such as the system 1000, may include a tip position detector 107 that operates to detect a position of the EM tracking sensor 106 and to output the detected positional information to the controller 102 (e.g., as shown in FIG. 5).

The controller 102 operates to receive the positional information of the tip of the continuum robot 104 from the tip position detector 107. The controller 102 operates to control the actuator 103 in accordance with the manipulation by a user (e.g., manually), or automatically (e.g., by a method or methods run by one or more processors using software, by the one or more processors, etc.) via one or more operation/operating portions or operational controllers 105 (e.g., such as, but not limited to a joystick as shown in FIG. 5). The one or more displays 101-1, 101-2 and/or operation portion or operational controllers 105 may be used as a user interface 3000 (also referred to as a receiving device) (e.g., as shown diagrammatically in FIG. 2). In an embodiment shown in FIG. 2 and FIG. 5, the system 1000 may include, as an operation unit, the display 101-1 (e.g., such as, but not limited to, a large screen user interface with a touch panel, first user interface unit, etc.), the display 101-2 (e.g., such as, but not limited to, a compact user interface with a touch panel, a second user interface unit, etc.) and the operating portion 105 (e.g., such as, but not limited to, a joystick shaped user interface unit having shift lever/button, a third user interface unit, a gamepad, or other input device, etc.).

The controller 102 may control the continuum robot 104 based on an algorithm known as follow the leader (FTL) algorithm. By applying the FTL algorithm, the middle section and the proximal section (following sections) of the continuum robot 104 may move at a first position in the same way as the distal section moved at the first position or a second position near the first position (e.g., during insertion of the continuum robot/catheter 104). Similarly, the middle section and the distal section of the continuum robot 104 may move at a first position in the same way as the proximal section moved at the first position or a second position near the first position (e.g., during removal of the continuum robot/catheter 104). Alternatively, the continuum robot/catheter 104 may be removed by automatically or manually moving along the same path that the continuum robot/catheter 104 used to enter a target (e.g., a body of a patient, an object, a specimen (e.g., tissue), etc.) using the FTL algorithm.

Any of the one or more processors, such as, but not limited to, the controller 102 and the display controller 100, may be configured separately. As aforementioned, the controller 102 may similarly include a CPU 120, a RAM 130, an I/O 140, a ROM 110, and a HDD 150 as shown diagrammatically in FIG. 3. Alternatively, any of the one or more processors, such as, but not limited to, the controller 102 and the display controller 100, may be configured as one device (for example, the structural attributes of the controller 100 and the controller 102 may be combined into one controller or processor, such as, but not limited to, the one or more other processors discussed herein (e.g., computer, console, or processor 1200, 1200′, etc.).

The system 1000 may include a tool channel for a camera, biopsy tools, or other types of medical tools (as shown in FIG. 5). For example, the tool may be a medical tool, such as an endoscope, a forceps, a needle or other biopsy tools, etc. In one or more embodiments, the tool may be described as an operation tool or working tool. The working tool may be inserted or removed through a working tool insertion slot 501 (as shown in FIG. 5).

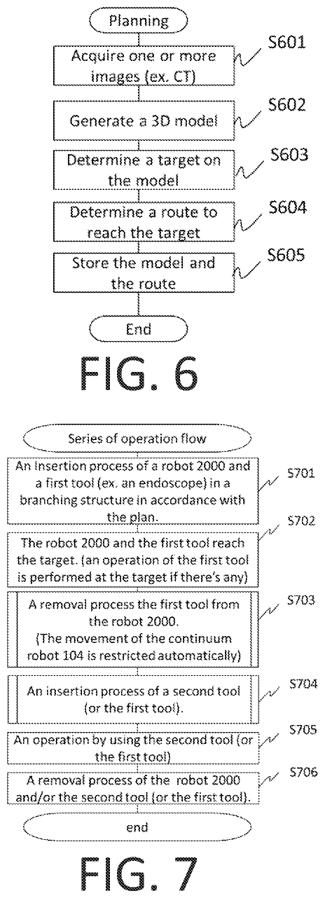

One or more of the features discussed herein may be used for planning procedures. As an example of one or more embodiments, FIG. 6 is a flowchart showing steps of at least one planning procedure of an operation of the continuum robot/catheter device 104. One or more of the processors discussed herein may execute the steps shown in FIG. 6, and these steps may performed by executing a software program read from a storage medium, including, but not limited to, the ROM 110 or HDD 150, by CPU 120 or by any other processor discussed herein. One or more methods of planning using the continuum robot/catheter device 104 may include one or more of the following steps: (i) In step 601, one or more images such, as CT or MRI images, may be acquired; (ii) In step 602, a three dimensional model of a branching structure (for example, an airway model of lungs or a model of an object, specimen or other portion of a body) may be generated based on the acquired one or more images; (iii) In step 603, a target on the branching structure may be determined (e.g., based on a user instruction, based on preset or stored information, etc.); (iv) In step 604, a route of the continuum robot/catheter device 104 to reach the target (e.g., on the branching structure) may be determined (e.g., based on a user instruction, based on preset or stored information, etc.); (v) In step 605, the generated three-dimensional model and the decided route on the model may be stored (e.g., in the RAM 130 or HDD 150, in any other storage medium discussed herein, in any other storage medium known to those skilled in the art, etc.). In this way, a 3D model of a branching structure may be generated, and a target and a route on the 3D model may be determined and stored before the operation of the continuum robot 104 is started.

In one or more of the embodiments below, embodiments of using a catheter device/continuum robot 104 are explained. However, an endoscope or other medical device (e.g., such as, but not limited to, a bronchoscope) may be used instead of the catheter device.

One or more of the features discussed herein may be used for a series of operation flow. By way of at least one embodiment example of a series of operation flow, FIG. 7 shows processes of an operation of the robot 2000 and at least one tool which is inserted into and/or removed from the robot 2000 (e.g., the continuum robot 104, the continuum robot 104 and the tracking sensor 106, inserted and removed via an opening such as the working tool insertion slot 501, etc.). These steps may be performed by any of the one or more processors discussed herein. For example, the steps of FIG. 7 or any other series of operation flow may be performed by executing a software program read from any storage medium discussed herein, including, but not limited to, the ROM 110 or HDD 150 (other examples, include, but not are not limited to, the memory 52, the storage 53, the ROM 1202, the RAM 1203, the hard disk 1204, the SSD 1207, etc. (as shown in one or more of FIGS. 16-17 and/or 20-21)), by the one or more processors, such as, but not limited to, the CPU 120 of the controller 102 or of the display controller 100 (or any other processors discussed herein (e.g., the display controller 100, the controller 102, the CPU 120, the controller 50, the CPU 51, the console or computer 1200 or 1200′, the CPU 1201, any other processor or processors discussed herein, etc.)).

One or more methods of planning using the continuum robot/catheter device 104 may include one or more of steps S701 through S706 as shown in FIG. 7. In an insertion process (S701), a robot 2000 and a first tool (for example, an endoscope, a camera, a catheter, a bronchoscope, etc.) may be inserted into a branching structure (for example, an airway of a patient) in accordance with the plan. In accordance with a proceeding or path of the actuator 103 and robot 2000 on or along the rail 108, the robot 2000 proceeds in the branching structure. By operating the operating portion or operational controller 105, a user (for example, a physician) may instruct the continuum robot 104 to bend so that the robot 2000 (or any portion thereof, such as, the continuum robot 104, the EM tracking sensor 106, the continuum robot 104 and the EM tracking sensor 106, etc.) reaches the target in the branching structure.

The robot 2000 and a first tool may be inserted into the object, such as the branching structure, independently or may be inserted at the same time. The robot 2000 and the first tool may be inserted into the target or the branching structure at the same time by inserting the robot 2000 with the first tool already being inserted in the robot 2000. For example, in one or more embodiments, the continuum robot 104 may be a catheter, and the first tool may be an endoscope. The endoscope may be set in the catheter and both the endoscope and the catheter 104 may be inserted into the object or the airway of a lung of a patient to reach a target in the lung. A physician (or other user) may control a posture of the catheter by operating or using the operating portion or operational controller 105 during the catheter and the endoscope process in, or path into, the airway. A captured image or images (e.g., a static image or a moving image) captured by the endoscope may be displayed on the one or more displays 101-1, 101-2, and the physician (or other user) may determine the posture of the catheter based on the displayed image or images.

After the robot 2000 and the first tool reach a target (S702), an operation by the first tool may be performed. That said, the operation of the first tool at the target is not necessarily performed. For example, in a case where the first tool is an endoscope, the endoscope may capture a static image of a target (for example, a nidus) or in a case where the first tool is a biopsy tool like needle or forceps, a sampling of a tissue may be performed by using the first tool at the target. Alternatively, in a case where the first tool is an endoscope, the endoscope may be only used for capturing images of a way or path from a start point to the target and any particular operation of the endoscope other than the operation of capturing the image or images of the way or path, or as the same as that of the way or the path, may not be performed.

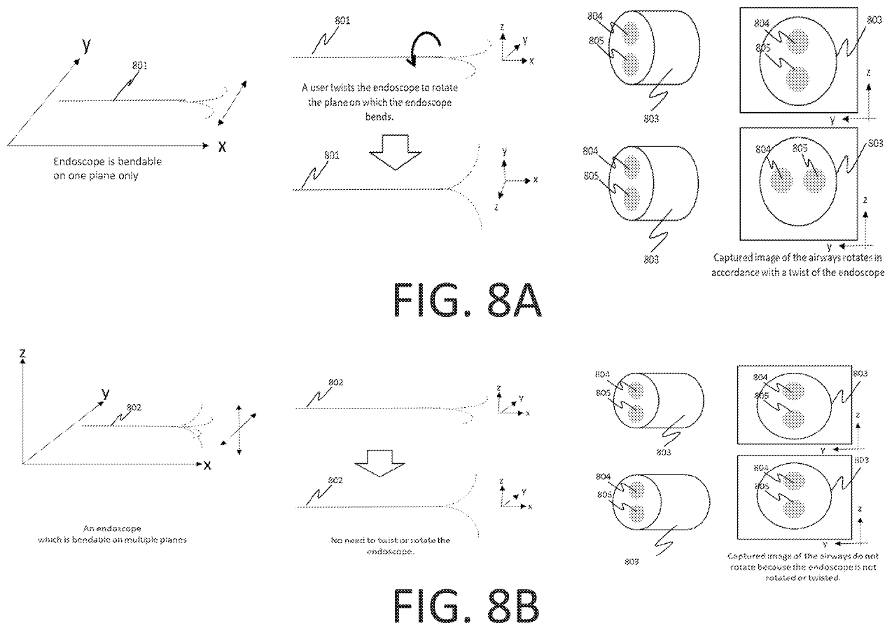

In a removal process (S703), the first tool may be removed from the robot 2000. A posture of movement of the continuum robot 104 may be restricted automatically in accordance with a removal of the first tool (e.g., the posture or position of the catheter 104 may remain the same while the first tool is removed from the robot 2000). For example, the endoscope may be removed from the catheter 104 after the endoscope and the catheter 104 reach the target. In a case where the removal of the endoscope is detected, the movement of the continuum robot 104 may be automatically locked. In this way, a positional relationship between the target and the tip of the catheter/continuum robot 104 may be kept or maintained during the removal of the endoscope from the catheter 104. At least one embodiment of a specific procedure in the removal process is explained below by using FIGS. 8A-8B.

In an insertion process (S704), a second tool (or the first tool) may be inserted into the robot 2000. For example, a biopsy tool (or a second tool) may be inserted into the catheter 104 after the endoscope (a first tool) is removed from the catheter 104. At least one embodiment of a specific procedure in the insertion process is also explained below by using FIGS. 8A-8B.

After the second tool (or the first tool) is inserted into the robot 2000, an operation of the inserted second tool (or the first tool) may be performed (S705). For example, in at least one embodiment, the first tool may be an endoscope and the second tool may be a biopsy tool. The endoscope (the first tool) may be removed from the catheter at S703, the biopsy tool (the second tool) may be inserted into the catheter at S704, and a biopsy operation may be performed by the biopsy tool at S705.

After the operation by using the second tool is finished, the second tool and the robot 2000 may be removed from the object or area (e.g., an area to be imaged, an area on which a plan is to be developed, an area for a medical procedure, etc.), such as the branching structure (S706).

In one or more embodiments of the present disclosure, rotation control of a captured image or images may be performed (e.g., rotation control may be performed using kinematics). By way of example of at least one embodiment, FIG. 8A shows a type of an endoscope 801 (also referred to herein as a second endoscope) which may bend on a single plane only. To change the bending direction from a horizontal direction to a vertical direction, a user twists or rotates the endoscope 801 by a predetermined amount, e.g., by 90 degrees. In accordance with the twisting or the rotation of the endoscope 801, an image or images captured by the endoscope 801 rotates. In FIG. 8A, before the endoscope 801 is rotated or twisted, an object or target area, such as a left airway 804 and right airway 805 of bronchus 803, are captured vertically. After the endoscope 801 is rotated or twisted, the object or the target area, e.g., the left airway 804 and the right airway 805, are captured horizontally. In one or more embodiments of the present disclosure, the endoscope 801 may be a conventional endoscope known to those skilled in the art, an endoscope that the physician or user of the endoscope is familiar or has experience with, an endoscope having the structure discussed herein, etc.

FIG. 8B shows an endoscope 802 (first endoscope) that operates to bend on any plane without rotation (e.g., the endoscope 802 may bend on multiple planes or on two or more planes). As shown in FIG. 8B, a layout of the airways 804 and 805 does not change because the endoscope 802 does not need to be rotated or twisted to change a bending direction.

As shown in FIGS. 8A and 8B, the layout of airways in a captured image of the same position on a lung sometimes may differ because of a difference or differences between endoscopes (e.g., the endoscope 801 is structurally and functionally different than the endoscope 802 as discussed above, the endoscopes are different types of endoscopes, the endoscopes include any other difference discussed herein, etc.). Such a difference or differences may make an experienced user confused when the user often uses the second endoscope 801 (e.g., an endoscope that is bendable on one plane only, an endoscope that is a conventional endoscope, an endoscope that the physician or user of the endoscope is familiar or has experience with, etc.) and the user newly uses the first endoscope 802 (e.g., an endoscope which is bendable on multiple planes, an endoscope which bends on two or more planes, etc.). In this embodiment or embodiments, an endoscope 802 (a first endoscope) which is bendable on any two or more planes is used to capture an image of inside of a target area, such as a lung (e.g., more than two (2) degrees of freedom (DoF)). In such embodiment(s), the captured image may be rotated so that the captured image of inside of the target area (e.g., the lung) is displayed as if the image was captured by an endoscope 801 (a second endoscope) which is bendable on a single plane (e.g., one (1) degree of freedom (DoF)) while the captured image is actually captured by the first endoscope 802. In one or more embodiments, the second endoscope 801 may be a virtual endoscope. A captured image that would have been captured by the second endoscope 801 may be theoretically obtained or estimated by the controller 102 (view controller) or any of the other one or more processors discussed herein.

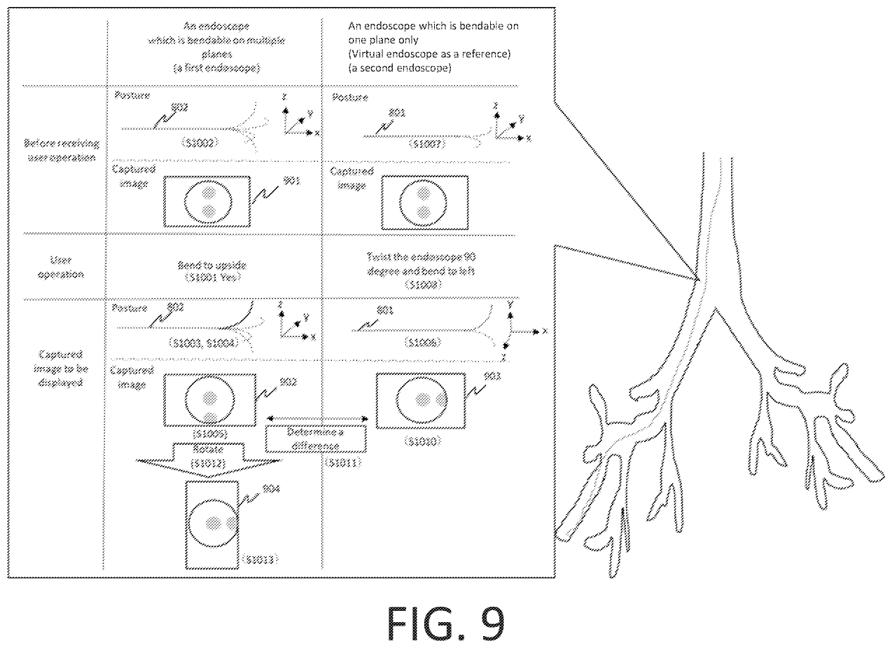

In the embodiment example shown in FIG. 9, the first endoscope 802 may be bent to upward, downward, right side and/or left side. On the other hand, the second endoscope 801 may be bent to right side and left side, and the second endoscope 801 cannot be bent to upward and downward directions. To move the tip of the second endoscope 801 to upward, a user may twist (e.g., clockwise, counter clockwise, etc.) the second endoscope 801 for a predetermined amount, e.g., 90 degrees, and to instruct to bend to the left, for example.

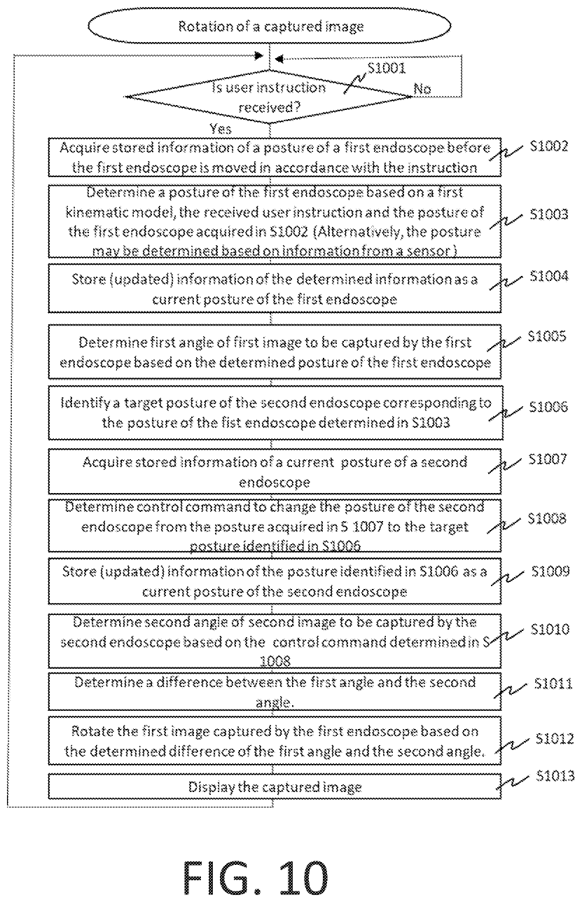

FIG. 9 and FIG. 10 show a rotation control of an image captured by the first endoscope 802. In at least this embodiment example, a first captured image captured by the first endoscope 802 may be rotated to match with a second captured image that is to be captured or that would be captured by the second endoscope 801 at the same position in a target area or object (e.g., in a lung, in a human body, in tissue, etc.). In at least this embodiment, a rotation control of a captured image captured by an endoscope is disclosed. The endoscope of this embodiment is an example of a continuum robot 104. The continuum robot 104 of this embodiment may be a combination of a catheter and an endoscope. In this case, a posture of an endoscope may be changed in accordance with a change of a posture or position of the catheter. According to at least the embodiment, a user may check the captured image of the inside of the target area or object (e.g., a lung, a human body, tissue, etc.) as if the user is using a second endoscope (e.g., the second endoscope 801, an endoscope different than the first endoscope, a conventional endoscope, an endoscope that the physician or user of the endoscope is familiar or has experience with, a conventional endoscope that is different than the first endoscope, an endoscope that only rotates in a single plane, etc.) while the user actually is using a different type of endoscope (e.g., a first endoscope that is different from the second endoscope, the first endoscope 802, an endoscope that rotates on two or more planes, etc.). In one or more embodiments, while not limited to this scenario, the second endoscope may be a conventional and accustomed type of endoscope, an endoscope that the user has used previously and is familiar with, etc.

One or more methods for performing a rotation of an image or images are discussed herein. By way of at least one embodiment example, FIG. 10 is a flowchart showing a method for performing a rotation of an image or images. The steps S1001-S1013 in FIG. 10 may be performed by any of the one or more processors discussed herein (e.g., the display controller 100, the controller 102, the CPU 120, the controller 50, the CPU 51, the console or computer 1200 or 1200′, the CPU 1201, any other processor or processors discussed herein, etc.). The steps in FIG. 10 may be performed by executing a software program read from any storage medium discussed herein, such as, but not limited to the ROM 110 or HDD 150 (other examples, include, but not are not limited to, the memory 52, the storage 53, the ROM 1202, the RAM 1203, the hard disk 1204, the SSD 1207, etc. (as shown in one or more of FIGS. 16-17 and/or 20-21)), by any processor discussed herein, such as, but not limited to, the CPU 120 of the controller 102 or of the display controller 100 (e.g., other processors may be the controller 50, the CPU 51, the console or computer 1200 or 1200′, the CPU 1201, any other processor or processors discussed herein, etc.). The controller 102 and the display controller 100 may be configured as one controller, or may be configured separately or independently.

In step S1001, the controller 102 and/or the display controller 100 (or other processor(s) discussed herein) may determine whether an instruction to move the endoscope has been received. If the instruction to move the endoscope has not been received (No in S1001), the controller 102 and/or the display controller 100 may repeat the process of step 1001. If the user instruction is received (yes in S1001), the controller 102 and/or the display controller 100 (or other processor(s) discussed herein) proceeds to S1002.

In S1002, the controller 102 and/or the display controller 100 (or other processor(s) discussed herein) may acquire from a memory (for example, the RAM 130, the RAM 1203, any other memory discussed herein, etc. (as shown in one or more of FIGS. 16-17 and/or 20-21)) stored information of a posture of a first endoscope 802 before the first endoscope 802 is moved in accordance with the received instruction.

In S1003, the controller 102 and/or the display controller 100 (or other processor(s) discussed herein) may determine a posture or position of the first endoscope 802 using a first kinematic model, the received user instruction, and the posture of the first endoscope 802 acquired in S1002. Alternatively, the posture may be determined based on positional information from a sensor (for example, EM tracking sensor 106, any other sensor or detector discussed herein, etc.). If the positional information from a sensor is used, steps from S1002 and S1004 may be omitted.

In S1004, the controller 102 and/or the display controller 100 (or other processor(s) discussed herein) may store in the memory determined information as a current posture or position of the first endoscope 802.

In S1005, the controller 102 and/or the display controller 100 (or other processor(s) discussed herein) may determine a first angle of at least a first image 902 to be captured based on the determined posture of the first endoscope 802.

In S1006, the controller 102 and/or the display controller 100 (or other processor(s) discussed herein) may identify a target posture or position of the second endoscope 801 corresponding to the posture or position of the first endoscope 802 determined in S1003.

In S1007, the controller 102 and/or the display controller 100 (or other processor(s) discussed herein) may acquire stored information of a current posture or position of a second endoscope 801.

In S1008, the controller 102 and/or the display controller 100 (or other processor(s) discussed herein) may determine commands or instructions to change the posture or position of the second endoscope 801 from the posture or position acquired in Slow to the target posture or position identified in S1006. For example, as shown in FIG. 9, if the first endoscope 802 receives a user operation to bend to upward, then the first endoscope 802 bends to upward. In S1008, the controller 102 and/or the display controller 100 (or other processor(s) discussed herein) may determine that the second endoscope 801 is needed to be twisted (e.g., clockwise, counter clockwise, etc.) for a predetermined amount (e.g., 90 degrees) and to be bent to the left so that the posture or position of the second endoscope 801 corresponds to the first endoscope 802.

In S1009, the controller 102 and/or the display controller 100 (or other processor(s) discussed herein) may store in any memory discussed herein information of the posture or position identified of the posture or position identified in S1006 as a current posture or position of the second endoscope 801.

In S1010, the controller 102 and/or the display controller 100 (or other processor(s) discussed herein) may determine a second angle of a second image 903 to be captured by the second endoscope 801 based on the control command or instruction determined in S1008. The second image 903 may be a theoretical image or an estimated image that would have been captured by the second endoscope 801. For example, the controller 102 and/or the display controller 100 (or other processor(s) discussed herein) may determine the second image 903 (an image after being rotated) based on the captured image before being rotated and the commands or instructions to change the posture or position of the second endoscope 801 (the commands or instructions may be, for example, “twist the second endoscope 90 degrees and bend to the left”).

In S1011, the controller 102 and/or the display controller 100 (or other processor(s) discussed herein) may determine a difference between the first angle and the second angle. In the example shown in FIG. 9, in S1011, the controller 102 and/or the display controller 100 (or other processor(s) discussed herein) may compare a first captured image 902 captured by a first endoscope 802 and a second captured image 903 to be captured by the second endoscope 801 when the posture or position of the second endoscope 801 is the same as the posture of the first endoscope 802. Then the controller 102 and/or the display controller 100 (or other processor(s) discussed herein) may determine that there is a difference of 90 degrees in an anticlockwise direction between the first endoscope 802 and the second endoscope 801. The difference between the first angle and the second angle may be determined based on a tilt angle of a coordinate 905 of the first endoscope 802 and a tilt angle of a coordinate 906 of the second endoscope 801. The x axis of the coordinate 905 and 906 corresponds to a longitudinal direction of the first endoscope 802 and the second endoscope 801. They axis of the coordinate 905 and 906 corresponds to right and left directions of the first endoscope 802 and the second endoscope 801. The z axis of the coordinate 905 and 906 corresponds to top and bottom directions of the first endoscope 802 and the second endoscope 801. The controller 102 and/or the display controller 100 (or other processor(s) discussed herein) may determine the difference between the first angle and the second angle based on a difference of a tilt angle of the y axis and/or the z axis of the coordinate 905 and coordinate 906. The tilt angles of the coordinates 905 and 906 may be determined based on the postures or positions of the first endoscope 802 and the second endoscope 801.

In S1012, the controller 102 and/or the display controller 100 (or other processor(s) discussed herein) may rotate the first image so that the rotated first image 904 corresponds to the second image 903.

In S1013, the controller 102 and/or the display controller 100 (or other processor(s) discussed herein) may display the rotated first image 904 on a display 101-1, 101-2.

In view of such feature(s) of the present disclosure, a user may check the captured image of the inside of the target area (e.g., a lung) as if the user is using the second endoscope (e.g., an endoscope different from the first endoscope, a conventional and accustomed type of endoscope, a conventional and accustomed type of endoscope that only bends in one plane, an endoscope that the physician or user of the endoscope is familiar or has experience with, etc.) while the user actually is using a different type of endoscope (e.g., a first endoscope). In one or more embodiments, a conventional and accustomed type of endoscope may be an endoscope that a user (e.g., a physician) has experience with, has used before, is familiar with, etc.

In one or more embodiments of the present disclosure, rotation control of a captured image or images may be performed (e.g., rotation control may be performed using pre-planned or predetermined data). As discussed with reference to one or more of the aforementioned embodiments, the posture or position of the second endoscope 801 and an angle of the image captured by the second endoscope 801 may be calculated based on the posture or position of the first endoscope 802. In one or more additional embodiments, an angle of images captured by the second endoscope 801 at each position of a target area (e.g., a lung, a portion of a human body, etc.) may be stored in advance.

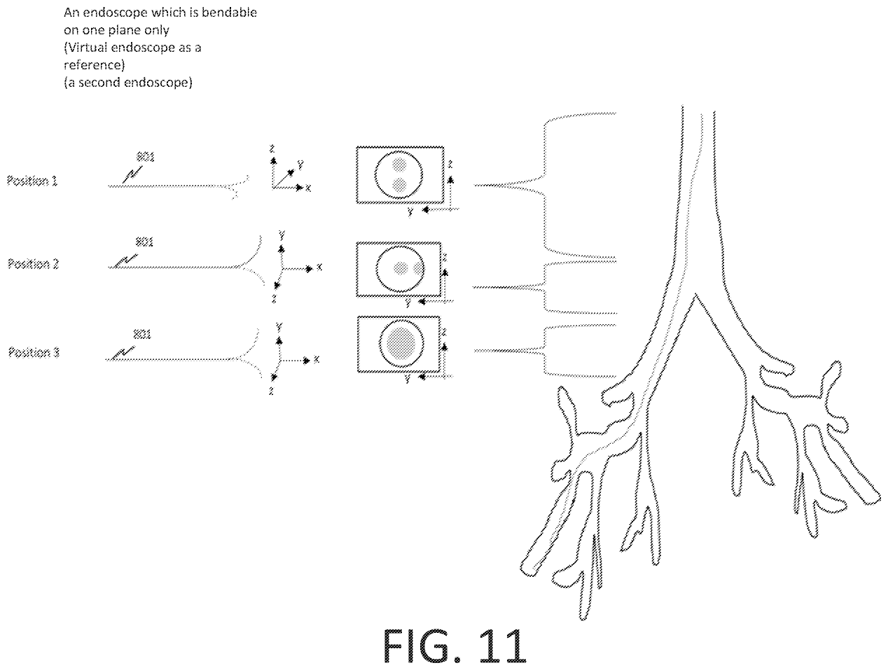

FIG. 11 shows at least one embodiment of planning of a route of the second endoscope 801 from a mouth to a target in a lung. In the planning process, a posture or position of the second endoscope 801 and angles of captured images captured by the second endoscope 801 at each positions on the route are simulated and stored.

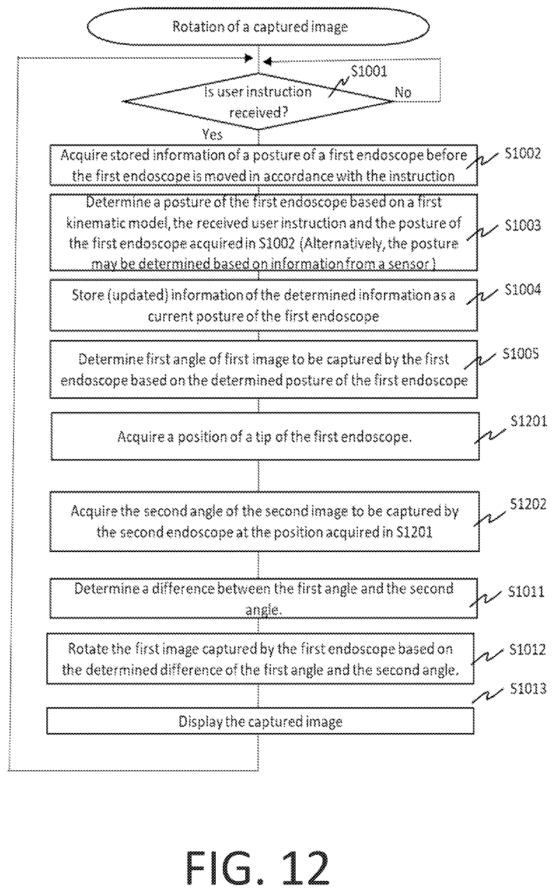

FIG. 12 shows a flowchart of at least one embodiment of a method of control of rotating a captured image or images. Steps S1001-S1013 in FIG. 12 may be performed by one or more processors discussed herein (e.g., the display controller 100, the controller 102, the CPU 120, the controller 50, the CPU 51, the console or computer 1200 or 1200′, the CPU 1201, any other processor or processors discussed herein, etc.). For example, the steps of FIG. 12 may be performed by executing a software program stored in one or more storage mediums discussed herein, such as, but not limited to, the ROM 110 or HDD 150 (other examples, include, but not are not limited to, the memory 52, the storage 53, the ROM 1202, the RAM 1203, the hard disk 1204, the SSD 1207, etc. (as shown in one or more of FIGS. 16-17 and/or 20-21)), by any processor(s) discussed herein, such as the CPU 120 of the controller 102 or of the display controller 100 (e.g., other processors may be the controller 50, the CPU 51, the console or computer 1200 or 1200′, the CPU 1201, any other processor or processors discussed herein, etc.). The controller 102 and the display controller 100 may be configured as one controller, or may be configured separately or independently as aforementioned. Because steps S1001 to S1005 and steps S1011 to S1013 are the same as what is explained above, the explanations for steps S1001 to S1005 and steps S1011 to S1013 are omitted in the subject discussion of FIG. 12. Steps 1201 to 1202 are discussed further below.

In step S1201, the controller 102 and/or the display controller 100 (or other processor(s) discussed herein) may acquire a position of a tip of the first endoscope 802.

In step S1202, the controller 102 and/or the display controller 100 (or other processor(s) discussed herein) may acquire the second angle of the second image to be captured by the second endoscope 801 at the position acquired in S1201. The second angle and the second image may be stored in a memory when the route of the second endoscope 801 is planned, in relation with a positional information of a tip of an endoscope. The controller 102 and/or the display controller 100 (or other processor(s) discussed herein) may acquire the second angle and the second image which is related to a positional information corresponding to the current position of the tip of the first endoscope.

In view of one or more features discussed in the present disclosure, a user (e.g., a physician) may check the captured image of the inside of a target area or object (e.g., the lung) as if the user is using the second endoscope (e.g., an endoscope different from the first endoscope, a conventional and accustomed type of endoscope, an endoscope that the physician or user of the endoscope is familiar or has experience with, a conventional and accustomed type of endoscope that only bends in one plane, etc.) while the user actually is using a different type of endoscope (e.g., a first endoscope). In one or more embodiments, a conventional and accustomed type of endoscope may be an endoscope that a user (e.g., a physician) has experience with, has used before, is familiar with, etc.

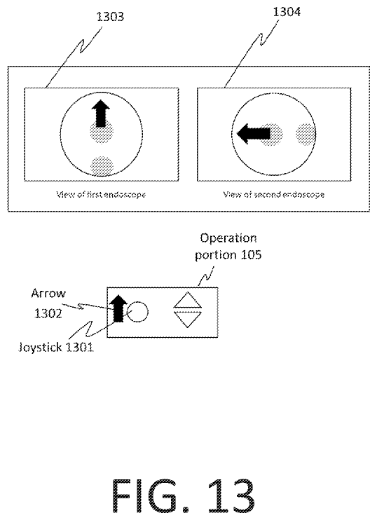

One or more embodiments may perform image processing on two views with a two (2) degree of freedom (DOF) controller. FIG. 13 shows an example of a display of a captured image 1303 captured by the first endoscope (e.g., the first endoscope 802) and a theoretical or estimated image 1304 that would have been captured by the second endoscope (e.g., the second endoscope 801) at the same position as a point where the first endoscope (e.g., the first endoscope 802) captures the captured image 1303. In one or more embodiments, the theoretical or estimated image 1304 is an additional image. The additional image may be obtained by one or more processors of the present disclosure, for example, by rotating the captured image 1303 as described above. Alternatively, the theoretical or estimated image 1304 may be displayed when a user instructed that such a display occur. Alternatively, the controller 102 or the display controller 100 (or other processor(s) discussed herein) may switch the image for display from the captured image 1303 to the theoretical or estimated image 1304 or from theoretical or estimated image 1304 to the captured image 1303 in accordance with a user instruction to switch the display of a captured image.

In one or more embodiments, a user may steer the first endoscope 802 based on the captured image 1303 (the view of the first endoscope 802). The direction to which a joystick 1301 (or other operational controller, such as, but not limited to the operating portion or operational controller 105) is tilted corresponds to a direction in the view of the first endoscope 802. For example, if a user tilts the joystick to upside or upwards (as shown via arrow 1302 in FIG. 13), then the captured image 1303 may be moved so that upside of the image comes to a center of the view and the theoretical or estimated image 1304 may be moved so that left side of the image comes to a center of the view.

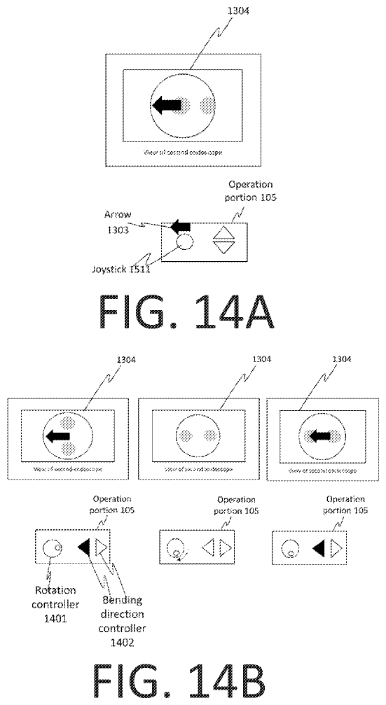

One or more embodiments may perform image processing on a single display with a two (2) degree of freedom (DOF) controller. In one or more of such embodiments, only the theoretical or estimated image 1304 may be displayed. In the example of FIG. 14A, though the theoretical or estimated image 1304 is displayed, operation of the operation portion or operational controller 105 corresponds to an operation of the second endoscope 801. The direction to which a joystick 1301 is tilted may correspond to a direction in the view of the second endoscope 801. For example, if a user tilts the joystick 1301 to the left (as shown via arrow 1303 in FIG. 14A), then the captured image 1304 may be moved so that left side of the image comes to a center of the view. The operation portion or operational controller 105 accepts information of an operation direction on the theoretical or estimated image 1304. The controller 100 or the controller 102 (or any other processor(s) discussed herein) may calculate the bending direction of the first endoscope 802 based on the accepted information and difference of an orientation of an xyz coordination shown in FIG. 9 between the first endoscope 802 and the second endoscope 801. In this way, a direction that the joystick 1301 is tilted and a directional command to bend the first endoscope 802 is adjusted.

While the user keeps tilting the joystick 1301 to send a series of commands, the rotation angle determined at S1012 may stay at the same angle at the beginning of the series of commands in order to avoid a huge rotation. At the completion of the series of commands, S1012 was applied to rotate the image. For example, if a user keeps tilting the joystick to the left, a series of commands is input to controller 102 and/or controller 100 (or any other processor(s) discussed herein). While the first endoscope 802 is bending in accordance with the input commands, the rotation of the view of the captured image may be restricted, and the theoretical or estimated image 1304 may not be rotated until the user stops tiling the joystick 1301. After the user stops tilting, the controller 102 or display controller 100 (or any other processor(s) discussed herein) may rotate the captured image based on the input commands and the theoretical image after rotated image or images is/are displayed. In this way, the controller 102 or the display controller 100 (or any other processor(s) discussed herein) may temporarily suspend the rotation of the captured image while the interface is receiving the command.

The controller 102 or the controller 100 (or any other processor(s) discussed herein) may rotate the image in accordance with each of the series of commands. For example, if the rotation angle determined at S1012 is larger than a predetermined threshold of the rotation angle, the predetermined angle may be applied to rotate the image. If the captured image is rotated too much, it may become difficult for a user to understand the direction where the endoscope currently faces, and the user may be confused. As such, in one or more embodiments, the optional restriction of the rotation angle may prevent the user from confusion, and may provide more accurate results.

One or more embodiments may perform image processing on a single display or view with a one (1) degree of freedom (DOF) controller. In the example of FIG. 14B, a user may input commands to operate an endoscope as if the user is steering the second endoscope 801, while the user is actually controlling the first endoscope 802. The direction to which a joystick 1301 (or operational controller 105) is tilted corresponds to a direction in the view of the second endoscope 801. For example, if a user tilts the joystick to left side, then the captured image 1303 may be moved so that left side of the image comes to a center of the view. The operation portion 105 shown in FIG. 14B may provide a user interface for a one (1) degree of Freedom (DOF) controlled endoscope (For example, an endoscope that only bends toward left and right). When a user wants to move the endoscope upward or downward in the theoretical or estimated image 1304, a user may rotate the rotation controller 1401 and the image rotates so that the left and right before rotation of the second endoscope 801 becomes upward and downward of the second endoscope 801 after being rotated. In such embodiments, a user may feel as if the user is rotating or twisting the second endoscope 801 by rotating the rotation controller 1401. However, actually, the user is steering the first endoscope 802 and the first endoscope is not rotated. Instead, the direction the first endoscope 802 bends in accordance with an operation of bending direction controller 1402 changes based on the rotation of the rotation controller 1401.

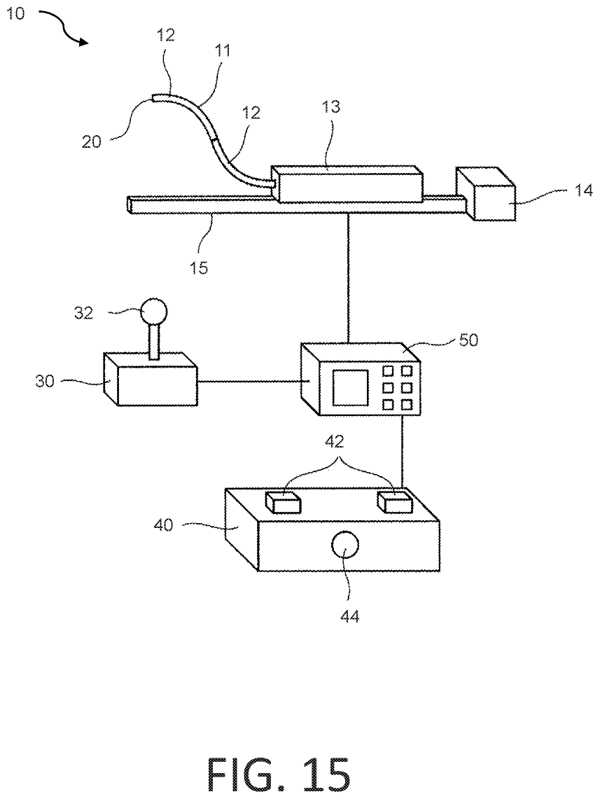

One or more of the aforementioned features may be used with a continuum robot and related features as disclosed in U.S. Provisional Pat. App. No. 63/150,859, filed on Feb. 18, 2021, the disclosure of which is incorporated by reference herein in its entirety. For example, FIGS. 15 to 17 illustrate features of at least one embodiment of a continuum robot apparatus 10 configuration to implement automatic correction of a direction to which a tool channel or a camera moves or is bent in a case where a displayed image is rotated. The continuum robot apparatus 10 enables to keep a correspondence between a direction on a monitor (top, bottom, right or left of the monitor) and a direction the tool channel or the camera moves on the monitor according to a particular directional command (up, down, turn right or turn left) even if the displayed image is rotated.

As shown in FIGS. 15 and 16, the continuum robot apparatus 10 may include one or more of a continuum robot 11, an image capture unit 20, an input unit 30, a guide unit 40, a controller 50, and a display 60. The image capture unit 20 can be a camera or other image capturing device. The continuum robot 11 can include one or more flexible portions 12 connected together and configured so they can be curved or rotated about in different directions. The continuum robot 11 can include a drive unit 13, a movement drive unit 14, and a linear drive 15. The movement drive unit 14 causes the drive unit 13 to move along the linear guide 15.

The input unit 30 has an input element 32 and is configured to allow a user to positionally adjust the flexible portions 12 of the continuum robot 11. The input unit 30 may be configured as a mouse, a keyboard, joystick, lever, or another shape to facilitate user interaction. The user may provide an operation input through the input element 32, and the continuum robot apparatus 10 may receive information of the input element 32 and one or more input/output devices, which may include a receiver, a transmitter, a speaker, a display, an imaging sensor, or the like, a user input device, which may include a keyboard, a keypad, a mouse, a position tracked stylus, a position tracked probe, a foot switch, a microphone, or the like. The guide unit 40 is a device that includes one or more buttons, knobs, switches, or the like 42, 44, that a user can use to adjust various parameters the continuum robot 10, such as the speed or other parameters.

FIG. 17 illustrates the controller 50 according to one or more aspects of the present disclosure. The controller 50 is configured to control the elements of the continuum robot apparatus 10 and has one or more of a CPU 51, a memory 52, a storage 53, an input and output (I/O) interface 54, and communication interface 55. The continuum robot apparatus 10 can be interconnected with medical instruments or a variety of other devices, and can be controlled independently, externally, or remotely by the controller 50.

The memory 52 may be used as a work memory. The storage 53 stores software or computer instructions. The CPU 51, which may include one or more processors, circuitry, or a combination thereof, executes the software developed in the memory 52. The I/O interface 54 inputs information from the continuum robot apparatus 10 to the controller 50 and outputs information for displaying to the display 60.

The communication interface 55 may be configured as a circuit or other device for communicating with components included the apparatus 10, and with various external apparatuses connected to the apparatus via a network. For example, the communication interface 55 may store information to be output in a transfer packet and output the transfer packet to an external apparatus via the network by communication technology such as Transmission Control Protocol/Internet Protocol (TCP/IP). The apparatus may include a plurality of communication circuits according to a desired communication form.

The controller 50 may be communicatively interconnected or interfaced with one or more external devices including, for example, one or more data storages, one or more external user input/output devices, or the like. The controller 50 may interface with other elements including, for example, one or more of an external storage, a display, a keyboard, a mouse, a sensor, a microphone, a speaker, a projector, a scanner, a display, an illumination device, or the like.

The display 60 may be a display device configured, for example, as a monitor, an LCD (liquid panel display), an LED display, an OLED (organic LED) display, a plasma display, an organic electro luminescence panel, or the like. Based on the control of the apparatus, a screen may be displayed on the display 60 showing one or more images being captured, captured images, captured moving images recorded on the storage unit, or the like.

The components may be connected together by a bus 56 so that the components can communicate with each other. The bus 56 transmits and receives data between these pieces of hardware connected together, or transmits a command from the CPU 51 to the other pieces of hardware. The components can be implemented by one or more physical devices that may be coupled to the CPU 51 through a communication channel. For example, the controller 50 can be implemented using circuitry in the form of ASIC (application specific integrated circuits) or the like. Alternatively, the controller 50 can be implemented as a combination of hardware and software, where the software is loaded into a processor from a memory or over a network connection. Functionality of the controller 50 can be stored on a storage medium, which may include RAM (random-access memory), magnetic or optical drive, diskette, cloud storage, or the like.

The units described throughout the present disclosure are exemplary and/or preferable modules for implementing processes described in the present disclosure. The term “unit”, as used herein, may generally refer to firmware, software, hardware, or other component, such as circuitry or the like, or any combination thereof, that is used to effectuate a purpose. The modules can be hardware units (such as circuitry, firmware, a field programmable gate array, a digital signal processor, an application specific integrated circuit or the like) and/or software modules (such as a computer readable program or the like). The modules for implementing the various steps are not described exhaustively above. However, where there is a step of performing a certain process, there may be a corresponding functional module or unit (implemented by hardware and/or software) for implementing the same process. Technical solutions by all combinations of steps described and units corresponding to these steps are included in the present disclosure.

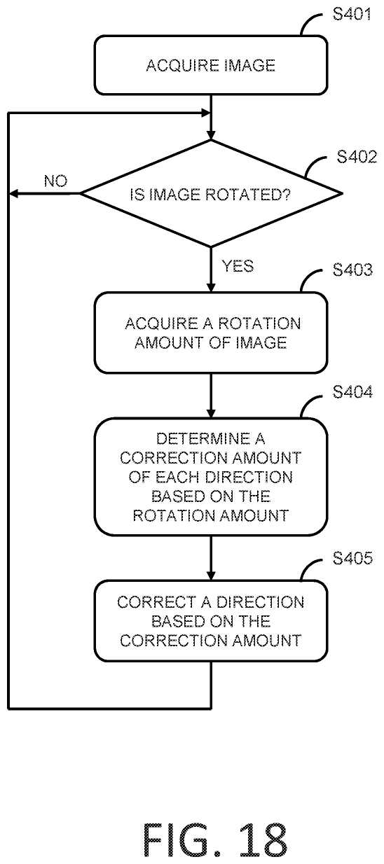

In one or more embodiments the medical tool may be a bronchoscope. Procedures for the automatic correction of the bending direction of a bronchoscope for one or more embodiments is illustrated in FIG. 18. The steps S401 to S405 shown in FIG. 18 may be performed by one or more computers, one or more processors, a central processing unit (CPU), for example, discussed herein. In step S401, a camera acquires an image. In step S402, the processor determines whether the camera image 100 displayed on the monitor 102 is rotated. The processor may determine that the camera image 100 is rotated in a case where rotation command of the camera image 100 is received. If the camera image 100 is not rotated, the processor repeats step S402. If it is determined that the camera image 100 is rotated, then the processor proceeds to step S403 and acquires a rotation amount of the camera image 100. For example, the rotation amount may be measured by an angle the camera image 100 is rotated on the monitor 102. In a case where the rotation amount is acquired, in step S403, the processor determines in step S404, based on the acquired rotation amount, a correction amount of each direction corresponding to respective moving commands (for example turn up, turn down, turn right or turn left command of the tip of the bronchoscope). Alternatively, in step S404, the processor determines, based on the acquired rotation amount, a correction amount of inclinations of axes. In step S405, the processor corrects, based on the result of the determination in step S404, each direction corresponding to respective moving commands or the inclinations of the axes. After the correction is performed, the process returns to step S402.

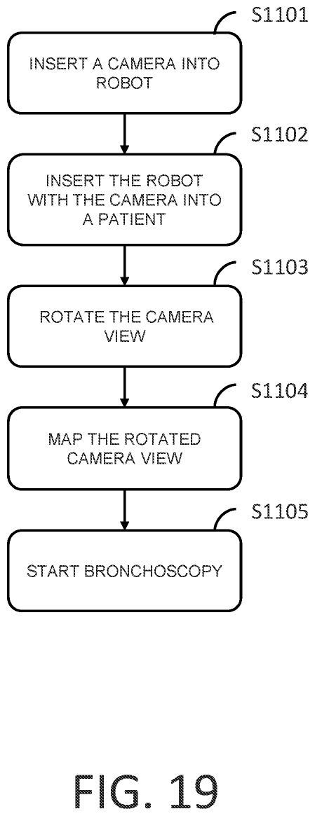

A flowchart for using a camera along with a bronchoscope is shown in FIG. 19. The detail of each step is as follows. Step S1101: an operator inserts a fiber optic camera into the robot through the tool channel, and deploys the camera at the tip of the robot. Step S1102: the operator inserts the robot with the camera into a patient through an endotracheal tube, and stops at the carina to display the right and left bronchus. At this point, the roll of the camera view may not be calibrated. Step S1103: the operator rotates the camera view until the right and left bronchus are displayed horizontally by adjusting the offset of the roll angle of camera view in software (offset 1). Step S1104: the operator maps the rotated camera view, the direction of joystick on the gamepad and the robot.

While the present invention has been described with reference to exemplary embodiments, it is to be understood that the invention is not limited to the disclosed exemplary embodiments. The scope of the claims is to be accorded the broadest interpretation so as to encompass all modifications, equivalent structures, and functions.

A computer, such as the console or computer 1200, 1200′, may perform any of the steps, processes, and/or techniques discussed herein for any apparatus and/or system being manufactured or used, any of the embodiments shown in FIGS. 1-19, any other apparatus or system discussed herein, etc.

There are many ways to control a continuum robot, correct or adjust an image, or perform any other measurement or process discussed herein, to perform continuum robot method(s) or algorithm(s), and/or to control at least one continuum robot device/apparatus, system and/or storage medium, digital as well as analog. In at least one embodiment, a computer, such as the console or computer 1200, 1200′, may be dedicated to control and/or use continuum robot devices, systems, methods, and/or storage mediums for use therewith described herein.

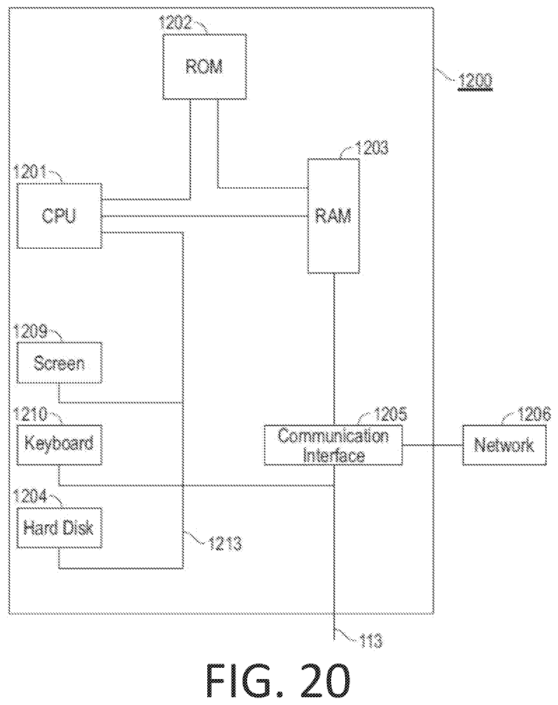

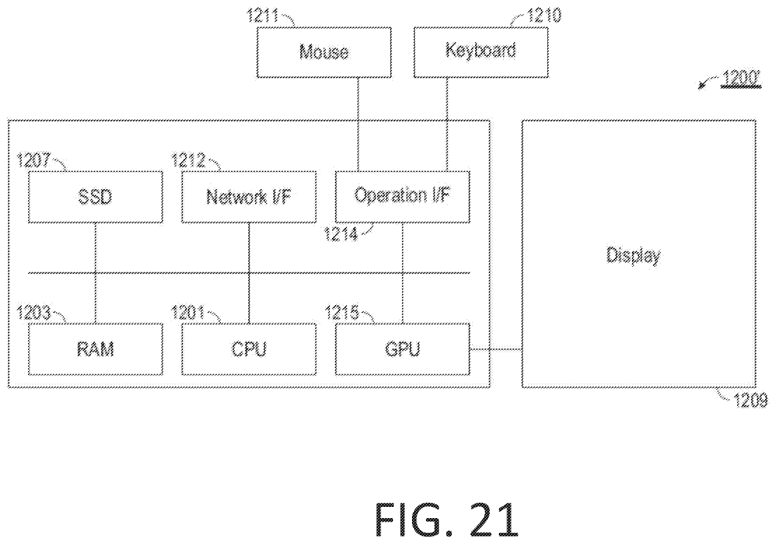

The one or more detectors, sensors, cameras, or other components of the apparatus or system embodiments (e.g. of the system 1000 of FIG. 1 or any other system discussed herein) may transmit the digital or analog signals to a processor or a computer such as, but not limited to, an image processor or display controller 100, a controller 102, a CPU 120, a controller 50, a CPU 51, a processor or computer 1200, 1200′ (see e.g., at least FIGS. 1-5, 15-17, and 20-21), a combination thereof, etc. The image processor may be a dedicated image processor or a general purpose processor that is configured to process images. In at least one embodiment, the computer 1200, 1200′ may be used in place of, or in addition to, the image processor or display controller 100 and/or the controller 102 (or any other processor or controller discussed herein, such as, but not limited to, the controller 50, the CPU 51, etc.). In an alternative embodiment, the image processor may include an ADC and receive analog signals from the one or more detectors or sensors of the system 1000 (or any other system discussed herein). The image processor may include one or more of a CPU, DSP, FPGA, ASIC, or some other processing circuitry. The image processor may include memory for storing image, data, and instructions. The image processor may generate one or more images based on the information provided by the one or more detectors, sensors, or cameras. A computer or processor discussed herein, such as, but not limited to, a processor of the devices, apparatuses or systems of FIGS. 1-5 and 15-17, the computer 1200, the computer 1200′, the image processor, etc. may also include one or more components further discussed herein below (see e.g., FIGS. 20-21).

Electrical analog signals obtained from the output of the system 1000 or the components thereof, and/or from the devices, apparatuses, or systems of FIGS. 1-6 and 15-17, may be converted to digital signals to be analyzed with a computer, such as, but not limited to, the computers or controllers 100, 102 of FIG. 1, the computer 1200, 1200′, etc.

As aforementioned, there are many ways to control a continuum robot, correct or adjust an image, or perform any other measurement or process discussed herein, to perform continuum robot method(s) or algorithm(s), and/or to control at least one continuum robot device/apparatus, system and/or storage medium, digital as well as analog. By way of a further example, in at least one embodiment, a computer, such as the computer or controllers 100, 102 of FIG. 1, the console or computer 1200, 1200′, etc., may be dedicated to the control and the monitoring of the continuum robot devices, systems, methods and/or storage mediums described herein.

The electric signals used for imaging may be sent to one or more processors, such as, but not limited to, the processors or controllers 100, 102 of FIGS. 1-5, a computer 1200 (see e.g., FIG. 20), a computer 1200′ (see e.g., FIG. 21), etc. as discussed further below, via cable(s) or wire(s), such as, but not limited to, the cable(s) or wire(s) 113 (see FIG. 20). Additionally or alternatively, the computers or processors discussed herein are interchangeable, and may operate to perform any of the feature(s) and method(s) discussed herein.

Various components of a computer system 1200 (see e.g., the console or computer 1200 as may be used as one embodiment example of the computer, processor, or controllers 100, 102 shown in FIG. 1) are provided in FIG. 20. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., as shown in FIG. 20). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a continuum robot device or system using same, such as, but not limited to, the system 1000, the devices/systems of FIGS. 1-6, and/or the systems/apparatuses of FIGS. 15-17, discussed herein above, via one or more lines 1213), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The computer system 1200 may include one or more additional processors in addition to CPU 1201, and such processors, including the CPU 1201, may be used for controlling and/or manufacturing a device, system or storage medium for use with same or for use with any continuum robot technique(s), and/or use with image correction or adjustment technique(s) discussed herein. The system 1200 may further include one or more processors connected via a network connection (e.g., via network 1206). The CPU 1201 and any additional processor being used by the system 1200 may be located in the same telecom network or in different telecom networks (e.g., performing, manufacturing, controlling, calculation, and/or using technique(s) may be controlled remotely).

The I/O or communication interface 1205 provides communication interfaces to input and output devices, which may include the one or more of the aforementioned components of any of the systems discussed herein (e.g., the controller 100, the controller 102, the displays 101-1, 101-2, the actuator 103, the continuum device 104, the operating portion or controller 105, the EM tracking sensor 106, the position detector 107, the rail 108, etc.), a microphone, a communication cable and a network (either wired or wireless), a keyboard 1210, a mouse (see e.g., the mouse 1211 as shown in FIG. 21), a touch screen or screen 1209, a light pen and so on. The communication interface of the computer 1200 may connect to other components discussed herein via line 113 (as diagrammatically shown in FIG. 20). The Monitor interface or screen 1209 provides communication interfaces thereto.

Any methods and/or data of the present disclosure, such as, but not limited to, the methods for using and/or controlling a continuum robot or catheter device, system, or storage medium for use with same and/or method(s) for imaging, performing tissue or sample characterization or analysis, performing diagnosis, planning and/or examination, controlling a continuum robot device or system, and/or for performing image correction or adjustment technique(s), as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 21), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, devices, systems, and computer-readable storage mediums related to the processors, such as, but not limited to, the processor of the aforementioned computer 1200, the processor of computer 1200′, the controller 100, the controller 102, etc., as described above may be achieved utilizing suitable hardware, such as that illustrated in the figures. Functionality of one or more aspects of the present disclosure may be achieved utilizing suitable hardware, such as that illustrated in FIG. 20. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 20 or FIG. 21, and/or which may be included in the computer, processor, controller and/or CPU 120 of FIGS. 1-5), CPU 51, and/or the CPU 120 may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 100, 102, 120, 50, 51, 1200, 1200′, etc.) may include the aforementioned CPU structure, or may be connected to such CPU structure for communication therewith.

As aforementioned, hardware structure of an alternative embodiment of a computer or console 1200′ is shown in FIG. 21. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid-state drive (SSD) 1207. Preferably, the computer or console 1200′ includes a display 1209 (and/or the displays 101-1, 101-2). The computer 1200′ may connect with one or more components of a system (e.g., the systems/apparatuses of FIGS. 1-5, 15-17, etc.) via the operation interface 1214 or the network interface 1212. The operation interface 1214 is connected with an operation unit such as a mouse device 1211, a keyboard 1210 or a touch panel device. The computer 1200′ may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 1200, the computer 1200′, etc.

At least one computer program is stored in the SSD 1207, and the CPU 1201 loads the at least one program onto the RAM 1203, and executes the instructions in the at least one program to perform one or more processes described herein, as well as the basic input, output, calculation, memory writing, and memory reading processes.

The computer, such as the computer 1200, 1200′, the computer, processors, and/or controllers of FIGS. 1-5 and/or FIGS. 15-17, etc., communicates with the one or more components of the apparatuses/systems of FIGS. 1-5, of FIGS. 15-17, and/or of any other system(s) discussed herein, to perform imaging, and reconstructs an image from the acquired intensity data. The monitor or display 1209 displays the reconstructed image, and may display other information about the imaging condition or about an object to be imaged. The monitor 1209 also provides a graphical user interface for a user to operate a system, for example when performing CT, MRI, or other imaging technique(s), including, but not limited to, controlling continuum robot devices/systems, and/or performing image correction or adjustment technique(s). An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs the system (e.g., the system 1000, the systems/apparatuses of FIGS. 1-5, the systems/apparatuses of FIGS. 15-17, any other system/apparatus discussed herein, etc.) to start or end the imaging, and/or to start or end continuum robot control(s) and/or performance of image correction or adjustment technique(s). The camera or imaging device as aforementioned may have interfaces to communicate with the computers 1200, 1200′ to send and receive the status information and the control signals.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Provisional Pat. App. No. 63/150,859, filed on Feb. 18, 2021, the disclosure of which is incorporated by reference herein in its entirety. Such endoscope devices, systems, methods, and/or storage mediums are disclosed in at least: U.S. patent application No. 17/565,319, filed on Dec. 29, 2021, the disclosure of which is incorporated by reference herein in its entirety; U.S. Pat. App. No. 63/132,320, filed on Dec. 30, 2020, the disclosure of which is incorporated by reference herein in its entirety; U.S. patent application No. 17/564,534, filed on Dec. 29, 2021, the disclosure of which is incorporated by reference herein in its entirety; and U.S. Pat. App. No. 63/131,485, filed Dec. 29, 2020, the disclosure of which is incorporated by reference herein in its entirety.

Although the disclosure herein has been described with reference to particular embodiments, it is to be understood that these embodiments are merely illustrative of the principles and applications of the present disclosure (and are not limited thereto), and the invention is not limited to the disclosed embodiments. It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications, equivalent structures, and functions.

1. An imaging device for performing image correction and/or adjustment, the device comprising:
one or more processors that operate to:
receive a captured image or images captured by a first imaging device at a first position;
obtain or determine an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device; and
adjust or correct the captured image or images based on the estimated image or images.

2. The imaging device of claim 1, further including a display to display the adjusted or corrected image or images.

3. The imaging device of claim 1, further comprising:
a receiver that operates to receive the captured image or images captured by the first imaging device at the first position, the first imaging device being a first endoscope, and transmit the capture image or images to the one or more processors such that the one or more processors receive the captured image or images;
a controller as being part of the one or more processors, the controller operating to obtain or determine the estimated image or images that would have been captured by the second imaging device at the first position, the second imaging device being a second endoscope; and
a display controller as being part of the one or more processors, the display controller or the one or more processors operating to provide an image or images for display, the image or images for display being based on the captured image or images and information for the estimated image or images.

4. The imaging device of claim 3,
wherein the second endoscope has a lower degree of freedom from a bendable degree of freedom of the first endoscope.

5. The imaging device of claim 3,
wherein the first endoscope is a camera deployed at a tip of a steerable catheter and is bent with the steerable catheter, and/or the camera is detachably attached to, or removably inserted into, the steerable catheter.

6. The imaging device of claim 3, wherein the second endoscope is a virtual endoscope or is represented by a preset or predetermined data profile.

7. The imaging device of claim 3, wherein the first endoscope has a bending section that operates to bend three-dimensionally and/or to bend on two or more planes.

8. The imaging device of claim 3, wherein the second endoscope has a bending section that can bend only on one plane.

9. The imaging device of claim 3,
wherein the image or images for display are the captured image or images that are rotated so that an orientation of the image or images for display corresponds to an orientation of the estimated image or images, and
wherein the display controller or the one or more processors display the image or images for display on a display.

10. The imaging device of claim 9, wherein the image or images for display comprise the captured image or images and an additional image or images, the additional image or images being the captured image or images that are rotated based on the information for the estimated image or images.

11. The imaging device of claim 10, wherein the captured image or images and the additional image or images are displayed at the same time.

12. The imaging device of claim 10, wherein the display controller or the one or more processors further operate to display the additional image or images in accordance with a user instruction to display the additional image or images on the display.

13. The imaging device of claim 10, wherein the display controller or the one or more processors further operate to switch the image or images for display from the captured image or images to the additional image or images.

14. The imaging device of claim 9, further comprising:
an interface configured to receive a command to bend the first endoscope,
wherein the display controller or the one or more processors further operate to temporarily suspend the rotation of the captured image or images while the interface is receiving the command.

15. The imaging device of claim 9, further comprising:
an interface configured to receive a command to bend the first endoscope,
wherein the display controller or the one or more processors further operate to restrict an amount of rotation of the captured image or images captured by the first endoscope in a case where the rotation angle of the captured image or images in accordance with the received command is larger than a predetermined rotation angle.

16. The imaging device of claim 9, further comprising:
an interface configured to receive a command to bend the first endoscope,
wherein the interface receives the command corresponding to a bending direction or a twisting amount of the second endoscope.

17. The imaging device of claim 3, further comprising:
a steerable catheter with at least one bending section and an endoscope camera; and
an actuation unit or a driver that operates to bend the bending section,
wherein the controller or the one or more processors further operate to:
receive one or more control commands or instructions for a bending amount and a bending plane orientation;
send the one or more commands or instructions to the actuation unit or the driver to bend the bending section; and
receive one or more endoscopic images and display the one or more endoscopic images on a display.

18. The imaging device of claim 17, wherein one or more of the following:
(i) the imaging device further comprises an operational controller or joystick that operates to issue or input the one or more commands or instructions to the controller or the one or more processors;
(ii) the imaging device further includes a display to display the one or more endoscopic images, or the imaging device further includes a display to display the one or more endoscopic images where the display has a reference direction;
(iii) the controller or the one or more processors further operate to store the bending plane orientation in the one or more control commands or instructions in relation to the one or more endoscopic images during navigation;
(iv) the controller or the one or more processors further operate to rotate a current endoscopic image to an orientation where a bending plane orientation stored at the last moment or stored last is aligned to a reference direction of a display of the imaging device; and/or
(v) the imaging device further comprises an operational controller or joystick that operates to issue or input the one or more commands or instructions to the controller or the one or more processors, and the operational controller or joystick operates to be controlled by a user of the imaging device.

19. The imaging device of claim 18, further comprising an operational controller or joystick, the operational controller or joystick having a rotation controller and a bending controller,
wherein one or more of the following:
the rotation controller operates to issue a control command or instruction of or for the bending plane orientation;
the bending controller operates to issue a control command or instruction of or for the bending amount; and/or
in a case where the controller or the one or more processors further operate to rotate a current endoscopic image to an orientation where a bending plane orientation stored at the last moment or stored last is aligned to a reference direction of a display of the imaging device, the controller or the one or more processors further operate to rotate the current endoscopic image as the rotation controller issues the control command or instruction of or for the bending plane orientation.

20. The imaging device of claim 3, further comprising:
a steerable catheter with at least one bending section and an endoscope camera;
an operational controller or joystick that operates to issue or input one or more commands or instructions of a bending amount and a bending plane orientation into the imaging device; and
a tracking device that operates to track a real-time bending plane orientation,
wherein the controller or the one or more processors operate to receive one or more endoscopic images and the one or more commands or instructions.

21. The imaging device of claim 20, wherein one or more of the following:
(i) the imaging device further comprises a display that operates to display the one or more endoscopic images;
(ii) the controller or the one or more processors further operate to recode the bending plane orientation in relation to the one or more endoscopic images during navigation; and/or
(iii) the imaging device further comprises a display that operates to display the one or more endoscopic images, the display having a reference direction, where the controller or the one or more processors further operate to rotate the current endoscopic image to an orientation where the real-time bending plane orientation from the tracking device is aligned to the reference direction of the display.

22. A method performing image correction and/or adjustment, the method comprising:
receiving a captured image or images captured by a first imaging device at a first position;
obtaining or determining an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device;
adjusting or correcting the captured image or images based on the estimated image or images; and
displaying the adjusted or corrected image or images on a display.

23. The method of claim 22, wherein the first imaging device is a first endoscopic device and the second image device is a second endoscopic device.

24. A non-transitory computer-readable storage medium storing at least one program for causing a computer to execute a method for performing image correction and/or adjustment, the method comprising:
receiving a captured image or images captured by a first imaging device at a first position;
obtaining or determining an estimated image or images that would have been captured using a second imaging device at the first position, the second imaging device having preset or predetermined navigation and/or viewing controls or orientations, and/or preset or predetermined structural features, that are different than navigation and/or viewing controls or orientations, and/or structural features, of the first imaging device;
adjusting or correcting the captured image or images based on the estimated image or images; and
displaying the adjusted or corrected image or images on a display.
